(19) United States
(12) Patent Application Publication
(10) Pub. No.: US 2025/0090242 A1
(43) Pub. Date: Mar. 20, 2025

(54) Registration of Medical Robot and/or Image Data for Robotic Catheters and Other Uses

(71) Applicant: Project Moray, Inc., Belmont, CA (US)

(72) Inventors: Erik Stauber, Albany, CA (US); Mark D. Barrish, Belmont, CA (US); Keith Phillip Laby, Oakland, CA (US); Steven Christopher Jones, Royston (GB); Mark Chang-Ming Hsieh, Cambridge (GB); Oliver John Matthews, Cambridge (GB)

(73) Assignee: Project Moray, Inc., Belmont, CA (US)

(21) Appl. No.: 18/899,409

(22) Filed: Sep. 27, 2024

Related U.S. Application Data
(63) Continuation of application No. PCT/US2023/016751, filed on Mar. 29, 2023.
(60) Provisional application No. 63/325,068, filed on Mar. 29, 2022.
(60) Provisional application No. 63/403,096, filed on Sep. 1, 2022.

(51) Int. Cl. A61B 34/20; A61B 6/00; A61B 6/12; A61B 6/46; A61B 8/00; A61B 8/08; A61B 8/12; A61B 34/30; A61B 90/00

(52) U.S. Cl. CPC A61B 34/20; A61B 6/12; A61B 6/463; A61B 6/487; A61B 6/5247; A61B 8/12; A61B 8/483; A61B 8/58; A61B 2034/2065; A61B 2034/301; A61B 2090/3966

(57) ABSTRACT

Devices, systems, and methods are provided for registering image data with robotic data for image-guided robotic catheters and other elongate bodies. Fluid drive systems can be used to provide robotically coordinated motion. Precise control over catheter-supported tools is enhanced by alignment of fluoroscopic and ultrasound image data, particularly for structural heart and other therapies in which the catheter will interact with soft tissues. A marker plate having a planar array of machine-readable 2D barcode markers may facilitate alignment of image and robotic data. Determining an ultrasound-based pose of a component in the ultrasound image field allows that component to effectively a fiducial for alignment.

### CROSS REFERENCE TO RELATED APPLICATIONS

The present application is a Continuation of PCT/US2023/016751 filed Mar. 29, 2023; which claims the benefit of U.S. Provisional Appln. Nos. 63/325,068 filed Mar. 29, 2022 and 63/403,096 filed Sep. 1, 2022; the disclosures which are incorporated herein by reference in their entirety for all purposes.

### FIELD OF THE INVENTION

In general, the present invention provides improved articulating devices, articulating systems, and methods for using elongate articulate bodies and other tools such as medical robots, cardiovascular catheters, borescopes, continuum robotics, and the like; as well as improved image processing devices, systems and methods that may find particularly beneficial use with such articulation and robotic technologies. In exemplary embodiments, the invention provides methods and systems that register robotic data used in control of a medical robot with ultrasound, fluoroscopic, and other image data, including such methods and systems configured to be used for display and image-guided movement of a portion of the medical robot that has been inserted into a patient.

### BACKGROUND OF THE INVENTION

Diagnosing and treating disease often involve accessing internal tissues of the human body, and open surgery is often the most straightforward approach for gaining access to those internal tissues. Although open surgical techniques have been highly successful, they can impose significant trauma to collateral tissues.

To help avoid the trauma associated with open surgery, a number of minimally invasive surgical access and treatment technologies have been developed. Interventional therapies are among the most successful minimally invasive approaches. An interventional therapy often makes use of elongate flexible catheter structures that can be advanced along the network of blood vessel lumens extending throughout the body. Alternative technologies have been developed to advance diagnostic and/or therapeutic devices through the trachea and into the bronchial passages of the lung. While generally limiting trauma to the patient, catheter-based endoluminal therapies can be challenging, in-part due to the difficulty in accessing (and aligning with) a target tissue using an instrument traversing a tortuous luminal path. Alternative minimally invasive surgical technologies include robotic surgery, and robotic systems for manipulation of flexible catheter bodies from outside the patient have also been proposed. Some of those prior robotic catheter systems have met with challenges, possibly because of the difficulties in effectively integrating large and complex robotic systems into clinical catheter labs, respiratory treatment suites, and the like. While the potential improvements to surgical accuracy make these efforts alluring, the capital equipment costs and overall burden to the healthcare system of these large, specialized systems is also a concern.

A range of technologies for controlling the shape and directing the movement of catheters have been proposed, including catheter assemblies with opposed pullwires for use in robotic articulating structures. Such structures often seek to provide independent lateral bending along perpendicular bending axes using two pairs of orthogonally oriented pullwires. As more fully explained in co-assigned PCT Publn. No. WO 2020/123671, which was filed on Dec. 11, 2019, and entitled “HYBRID-DIMENSIONAL, AUGMENTED REALITY, AND/OR REGISTRATION OF USER INTERFACE; AND SIMULATION SYSTEMS FOR ROBOTIC CATHETERS AND OTHER USES,” the full disclosure of which is incorporated herein by reference, new fluid-driven and other robotic catheter systems can optionally be driven with reference to a 3D augmented reality display. While those advantageous drive systems, articulation control, and therapy systems will find a wide variety of applications for use by interventional and other doctors in guiding the movement of articulated therapy delivery systems within a patient, it would be beneficial to even further expand the capabilities of these compact and intuitive robotic systems.

Precise control of both manual and robotic interventional articulating structures can be complicated not only by the challenges of accessing the target tissues through the bends of the vasculature, but also by the additional challenges of supporting those articulating structures within a therapy site and accurately identifying their position, orientation, and articulation state within that site, particularly when that site is surrounded by the delicate tissues and physiological movement of the cardiovascular system. Excessively rigid support structures within the vasculature could be difficult to introduce and may cause collateral tissue trauma. Position, orientation, and articulation state sensor systems can add complexity, size, and expense, limiting the number of patients that could benefit from new structural heart and other interventional therapies. Even seeing the articulating structures within the therapy site can present challenges, as optical imaging through the blood within the cardiovascular system is typically limited or unavailable. Interventionalists often rely on multiple remote imaging modalities to plan and guide different aspects of the therapy, for example, viewing single-image-plane fluoroscopy images while accessing the heart and then viewing multi-plane or 3D echocardiography images to see and interact with the target tissue structures. Maintaining situational awareness and precise control of a complex interventional therapy in this environment can be a significant challenge.

In general, it would be beneficial to provide improved medical robotic and other articulating devices, systems, and methods. It would be particularly beneficial if these improved technologies could expand the capabilities and ease-of-use of image guidance systems for use in diagnostic and therapeutic interventional procedures, ideally by providing registration technologies which help register the robotic data used to control movement of structures within the patient with the display images (such as ultrasound images, fluoroscope images, and the like) presented to the clinical user, as well as with the movement input commands from the user to the system. Such registration technologies may, for example, provide automated or semi-automated alignment between a position and orientation of a robotic structural heart therapy catheter and a heart valve tissues (both as shown in an echocardiography image shown to the clinical user) and a movement command input by the clinical user. Registration of the robotic and image data may allow, for example, the robotic system to move the end of the catheter up and to the right in the image by 1 cm, with a clockwise rotation of the end of the catheter by 20 degrees, to generate movement of the robotic catheter system so that the image of the tip of the catheter, as shown to the user in the display, moves up and to the right in the image by approximately 1 cm, with a clockwise rotation of the end of the catheter by approximately 20 degrees.

### BRIEF SUMMARY OF THE INVENTION

The present invention generally provides improved registration of medical imaging and robotic or other articulating devices, systems, and methods. Exemplary devices, systems, and methods are provided for guiding or controlling tools for interventional therapies which are guided with reference to an echo image plane or “slice” through the worksite. Optionally, interventional guidance systems employs a multi-thread computer vision or image processing architecture with parent/child reference frames, particularly for complex interventional therapies making use of multiple image modalities and/or that have multiple tool components in which one of the components articulates or moves relative to another. In some embodiments, devices, systems, and methods are provided for registering image data with robotic data for image-guided robotic catheters and automated control of other elongate bodies. Fluid drive systems can optionally be used to provide robotically coordinated motion. Precise control over actual robotic catheter-supported tools is enhanced by alignment of the robotic control workspace with fluoroscopic and ultrasound image data, particularly for robotic systems used for structural heart and other therapies in which the catheter will interact with soft tissues bordering a chamber of the heart. A marker plate having a planar or multi-planar array of machine-readable 2D barcode markers formed from tantalum or another high-Hounsfield unit material may facilitate alignment of the fluoroscope image data and the robotic data. An articulated catheter may be advanced to the chamber of the heart through a guide sheath. The guide sheath may have a machine-identifiable guide marker near its distal end. A data processor system may, in response to the fluoroscope image data, identify a pose (including a position and orientation) of the guide within the chamber, ideally relative to the marker plate. The data processor may also determine a pose of an ultrasound image probe, such as a Transesophageal Echocardiography (TEE) or Intracardiac Echocardiography (ICE) image probe in or near the chamber from the fluoroscope image data, optionally using markers mounted to the probe or an intrinsic image of the echo probe. One, two, or more echo image plane pose(s) relative to the marker plate may be determined using the probe pose. Determining an ultrasound-based pose of a robotic toolset component in the ultrasound image field allows that component to be aligned with the component pose in the fluoroscopic image data and/or the robotic data, effectively using the robotic component as a fiducial for alignment.

In a first aspect, the invention provides a medical system for a user to diagnose or treat a patient. The system may be for use with an ultrasound imaging probe, a fluoroscopy system, and a toolset. The probe can be insertable into a patient body and the toolset may have a proximal end and a distal end with an axis therebetween, the distal end insertable into the patient body. The medical system comprises a data processor having an ultrasound input for receiving ultrasound image data generated using the probe. The ultrasound image data may encompass a distal portion of the toolset and a target tissue of the patient within an ultrasound image field. A fluoroscopy input may be configured for receiving fluoroscopy image data generated using the fluoroscopy system, and the fluoroscopy image data may encompass the distal portion of the toolset and the probe within a fluoroscopy image field. The ultrasound image data and the fluoroscopy image data may comprise image data. A pose determining module may be coupled with the ultrasound input and the fluoroscopy input, the pose determining module configured for determining, in response to the image data, a pose of the toolset within the patient body. A display may be coupled with the processor so as to display an image of the toolset, in response to the pose data, such that the user can guide diagnosis or treatment of the target tissue.

A number of optional features may be included, combined, or used separately with the systems, devices, and methods described herein. For example, the processor may comprise a data processor for a multi-image-mode and/or a multi-component interventional system. The toolkit may comprise a first interventional component configured for insertion into an interventional therapy site of the patient body. The ultrasound input may comprise a first input for receiving a first image data stream of the interventional therapy site, the first image data stream including image data of the first component. The fluoroscopy input may comprise a second input for receiving a second image data stream. Optionally, the processor comprises a first module, a second module, and an alignment module. The first module may be coupled to the first input, the first module determining a pose of the first interventional component relative to the first image data stream in response to the image data of the first component. The second module may be coupled to the second input, the second module determining an alignment of the first image data stream relative to the patient body in response to the second image data stream. The alignment module may be configured for determining pose data of the first component relative to the patient body from the pose of the alignment of the first image data stream and from the pose of the first interventional component relative to the first image data stream. The output can be configured for transmitting interventional guiding image data and the pose data of the first component relative to the patient data.

In another aspect a data processor for a multi-image-mode and/or a multi-component interventional system. The system comprises a first interventional component configured for insertion into an interventional therapy site of a patient body having a target tissue. A first input is configured for receiving a first image data stream of the interventional therapy site. The first image data stream includes image data from the first component. A second input is configured for receiving a second image data stream. A first module is coupled to the first input, the first module determining a pose of the first interventional component relative to the first image data stream in response to the image data of the first component. A second module is coupled to the second input, the second module determining an alignment of the first image data stream relative to the patient body in response to the second image data stream. A registration module is configured for determining pose data of the first component relative to the patient body from the pose of the first image data stream and from the alignment of the first interventional component relative to the first image data stream. An output may transmit interventional guiding image data including the pose data of the first component relative to the patient data.

A number of independent features and refinements may optionally be included in the structures and methods described herein. For example, the first image data stream and/or the second image data stream may optionally comprise an ultrasound image data stream, often comprising a planar echo image. The first module may optionally determine the pose data of the first interventional component relative to a 3D image data space of the ultrasound image data stream, with that 3D space typically being defined by a location of an ultrasound transducer and/or electronic steering of image planes relative to that transducer. Typically, the first image data stream comprises tilt sweep data defined by a series of image planes extending from a surface of a transesophageal echocardiography (TEE) transducer surface, and by tilt angles between the planes and the TEE transducer surface (which can be varied electronically). To obtain the component pose data relative to a 3D ultrasound image space, the first module can be configured to extract still frames associated with the planes. Those still frames can be assembled into a 3D point cloud of data, with the relevant portions of the data being identified as exceeding a threshold. A number of image processing techniques can be applied to derive the component pose data, including generating of a 3D mesh and a 3D skeleton from the 3D point cloud, fitting of model sections based on the mesh and the skeleton, and fitting of a curve to the model sections to determine the pose data of the first component, particularly where the first component comprises a cylindrical catheter body having a bend.

Optionally, the first component has a machine-readable marker and the second image data stream comprises a fluoroscopic image data stream including image data of the marker. The second image data stream may also include image data of a pattern of machine-identifiable fiducial markers, the fiducial markers included in a marker board supported by an operating table. The second module can be configured to determine the pose data by determining a fluoroscope-based pose of the first component in response to the image data of the marker and the pattern of machine-identifiable fiducial makers, and by comparing the pose of the component from the ultrasound image data stream with the fluoroscope-based pose of the first component. The pose data may comprise a pose of the first component relative to the operating table (and hence the patient body), or may be indicative of a confidence of the fluoroscope-based pose.

The technologies described herein are suitable for integration of image data acquired via different image modalities having different capabilities. For example, the first image data stream can be generated by a first image capture device having a first imaging modality, with the component being more distinct in the first image data stream and the target tissue being indistinct (relatively speaking) in the first image data stream, such as when viewing such structures during a structural heart therapy in a fluoroscope image. The second image data stream may, in contrast, be generated by a second image capture device having a second imaging modality, the component being relatively indistinct in the second image data stream (as compared to in the first image data stream) and the target tissue being relatively distinct in the second image data stream (such as with the use of ultrasound data). With the use of a fluoroscope image data stream, including a machine-readable marker in the first component can provide significant advantages, as image processing techniques which identify the marker (and the associated first component), its location, and its orientation can be employed, with the use of a pattern of off-the-shelf markers (such as Aruco markers) or custom markers providing robust and accurate localization capabilities. Optionally, the second image data stream may comprise the same fluoroscopic image data stream as the first image data stream, with the fluoroscope image data including image data of one or more marker included on a second component. The first and second modules may comprise first and second computer vision software threads configured for identifying first and second pose data regarding the first and second components, respectively, with the threads optionally running relatively independently in separate cores of the processor on this common image stream. A similar multi-thread architecture to determine parent and child pose data can have a wide range of beneficial applications. For example, the first interventional component may optionally comprise a robotic steerable sleeve, and the second component may, for example, comprise a guide sheath having a lumen, with the lumen receiving the steerable sleeve axially therein. This will allow the steerable sleeve to be driven accurately relative to the patient's tissues by monitoring the pose of the sleeve relative to the guide sheath, facilitating the use of a flexible guide sheath (which will ideally be stiffer than the steerable sleeve) as a base for that movement despite the guide sheath itself also moving to some extent with physiological movement of the surrounding tissue.

A variety of alternative multi-thread image processing modules with parent/child relationships between their associated reference frames may be provided and optionally combined together. For example, the second component may optionally comprise a TEE or ICE probe. A probe system comprising the TEE or ICE probe and an ultrasound system will often generate image steering data indicative of alignment of the second image data stream relative to a transducer of the TEE or ICE probe. The pose data of the first component can be generated using the image steering data. Optionally, an optical character recognition module can be included for determining the image steering data from the second image data stream. Alternatively, such electronic image steering data may be transmitted more directly between the ultrasound system and the data processor.

In another aspect, the invention provides a method for using a medical robotic system to diagnose or treat a patient. The method comprises receiving fluoroscopic image data with a data processor of the medical robotic system, the fluoroscopic image data encompassing a portion of a toolset of the medical robotic system within a therapy site of the patient. Ultrasound image data is also received with the data processor, the ultrasound image data encompassing the portion of the toolset and a target tissue of the patient within an ultrasound image field. The data processor determines, in response to the image data, a pose of the toolset within the ultrasound image field. For example, the processor may optionally, in response to the fluoroscopic image data, determine an alignment of the toolset with the therapy site, and may also determine, in response to the ultrasound image data, an ultrasound-based pose of the toolset within the ultrasound image field. The data processor transmits, in response to a desired movement of the toolset relative to the target tissue in the ultrasound image field and the pose, a command for articulating the toolset at the therapy site so that the toolset moves per the desired movement.

A number of refinements may optionally be included for each of the aspects of the invention provided herein, with these refinements being included independently or in advantageous combinations to enhance the functionality of the inventions. For example, the data processor may optionally calculate, in response to the ultrasound-based pose and the alignment, a registration of the ultrasound image field with the therapy site. Typically, the transmitted command is determined by the data processor using the registration.

In another aspect, the invention provides a method for using a medical system to diagnose or treat a patient, the method comprising receiving a series of planar ultrasound image datasets with a data processor of the medical system. The ultrasound image datasets may encompass or define a series of cross-sections of a portion of the toolset of the medical system and a target tissue of the patient within an ultrasound image field. The data processor may determine, in response to the ultrasound image datasets, an ultrasound-based pose of the toolset within the ultrasound image field. The data processor may also transmit, in response to the ultrasound-based pose, a composite image including ultrasound imaging with a model of the toolset. The composite image can be transmitted so that it is displayed by a user of the medical system.

In another aspect, the invention provides a method for using a medical robotic system to diagnose or treat a patient. The method comprises calibrating fluoroscopic image data generated by a fluoroscopic image acquisition system. A processor of the medical robotic system may determine, in response to the calibrated fluoroscopic image data, an alignment of the fluoroscopic image data with a therapy site by imaging the therapy site and a plurality of fiducial markers using the fluoroscopic image acquisition system. The fluoroscopic image acquisition system may capture toolset image data encompassing a portion of a toolset of the medical robotic system in the therapy site. The processor of the medical robotic system may calculate, in response to the captured toolset image data and the determined alignment, a pose of the toolset in the therapy site. Movement of the toolset may be driven in the therapy site using the pose.

Optionally, the captured toolset image data does not include some or all of the fiducial markers. In fact, some or all of the fiducial markers may be intentionally displaced from a field of view of the fluoroscopic image acquisition system between i) the imaging of the therapy site and the fiducial markers, and ii) the capturing of the toolset image data. The portion of the toolset imaged may comprises a guide sheath having a lumen extending from a proximal end outside the patient distally to the therapy site. The pose may comprise a pose of the guide sheath, which optionally comprises a non-articulated guide sheath. Movement of the toolset may be performed by axially and/or rotationally moving a shaft of the toolset through the guidesheath. Movement of the toolset may comprise articulating a steerable body of the toolset extending through the lumen while the guide sheath remains in the pose.

An image capture surface of the fluoroscopic image acquisition system may optionally be disposed above the patient during use. Somewhat surprisingly, the determining and calculating steps may be performed using an optical acquisition model having a model image acquisition system disposed below the patient during use. To improve performance of the system, the processor of the medical robot system may superimpose models of the fiducial markers on the imaged therapy site based on the alignment. The superimposed models of the fiducial markers may be compared to the imaged fiducials of the fluoroscopic image data. Based on that comparison, the data processor may determine an error of the alignment, and may compensate for the error of the alignment. For example, the processor may, in an image of the therapy site displayed to the user, compensate for the error so that a model of the toolset portion superimposed on the imaged therapy site and an image of the toolset substantially correspond in the image of the therapy site. Optionally, the processor may superimpose a model of one or more component of the toolset on an image encompassing the toolset, compare the image of the toolset to the superimposed model toolset, and determine an error based on the comparison, and compensate for the error. Other uses of the error may include, with or without compensation for the error and displaying compensated images, calculating a confidence level for the alignment or pose and (optionally) displaying indicia of that confidence level or a suggestion that the user take some action such as re-alignment or re-registering the system.

Preferably, the fiducial markers comprise machine-readable standard 1D or 2D barcode markers, such as Aruco markers, QR codes, Apriltags, or the like. Alternatively, the fiducial markers may comprise custom machine-readable 1D or 2D fiducial barcode markers, such as VuMark™ makers which can be generated using software available from Vuforia, or any of a variety of alternative suppliers. Advantageously, these markers may be automatically identified, and codes of the fiducial markers can be read with the processor of the robotic system in response to the image data, and those codes can be used to help determine the alignment, particularly using models of the fiducial markers and/or of the toolset stored by the processor system. Hence, the toolset may have one or more toolset fiducial markers comprising one or more machine-readable standard or custom 2D fiducial barcode marker. The processor may automatically identify the one or more codes of the toolset fiducial marker(s) in response to the image data, and may using the toolset code(s) to determine the alignment. Along with determining the alignment, such machine-readable markers may be used to track changes in alignment of the therapy site and or toolset based on the image data.

In another aspect, the invention provides a medical robotic system for diagnosing or treating a patient. The system comprises a toolset having a proximal end and a distal end with an axis therebetween. The toolset is configured for insertion distally into a therapy site of the patient. The system also comprises a data processor having: i) a fluoroscopic data input for receiving fluoroscopic image data encompassing a portion of a toolset of the medical robotic system within the therapy site; ii) an ultrasound data input for receiving ultrasound image data encompassing the portion of the toolset and a target tissue of the patient within an ultrasound image field; iii) an alignment module for determining, in response to the fluoroscopic image data, an alignment of the toolset with the therapy site; iv) an ultrasound-based pose module for determining, in response to the ultrasound image data, an ultrasound-based pose of the toolset within the ultrasound image field; and v) an input for receiving a desired movement of the toolset relative to the target tissue in the ultrasound image field. A drive system may couple the toolset with the data processor. The data processor may be configured to, in response to the desired input, the alignment, and the ultrasound-based pose, transmit a command to the drive system so that the drive system moves per the desired movement.

In yet another aspect, the invention provides a medical system to diagnose or treat a patient. The system comprises a data processor having: i) an ultrasound input for receiving a series of planar ultrasound image datasets encompassing a series of cross-sections of a portion of the toolset of the medical system and a target tissue of the patient within an ultrasound image field; ii) an ultrasound-based pose determining module for determining, in response to the ultrasound image datasets, an ultrasound-based pose of the toolset within the ultrasound image field; and iii) an image output for transmitting, in response to the ultrasound-based pose, composite image data. A display may be coupled with the image output so as to display, in response to the composite image data transmitted from the image output, an ultrasound image with a model of the toolset superimposed thereon.

In yet another aspect, the invention provides a medical robotic system to diagnose or treat a patient. The system comprises a robotic toolset having a proximal end and a distal end with an axis therebetween. The distal end is configured for insertion into an internal therapy site of the patient. A plurality of fiducial markers are configured to be supported near the internal therapy site. A data processor has: i) a calibration module for calibrating fluoroscopic image data generated by a fluoroscopic image acquisition system; ii) an alignment module for determining, in response to the calibrated fluoroscopic image data encompassing the therapy site and at least some of the fiducial markers, an alignment of the fluoroscopic data with the robotic data; iii) a fluoroscopic toolset data input for receiving fluoroscopic toolset image data encompassing a portion of a toolset of the medical robotic system in the therapy site; and iv) a pose module for calculating, in response to the captured toolset image data and the determined alignment, a pose of the toolset in the therapy site. A drive system may couple the data processor with the robotic toolset so that the drive systems induce movement of the toolset in the therapy site using the pose.

In yet another aspect, the invention provides an interventional therapy or diagnostic system for use with a fluoroscopic image capture device for treating a patient body. The system comprises an elongate flexible body having a proximal end and a distal end with an axis therebetween. The elongate body is relatively radiolucent and can be configured to be advanced distally into the patient body. First and second machine-readable radio-opaque markers may be disposed on the elongate flexible body, the markers having first and second opposed major surfaces. The first major surfaces may be oriented radially outwardly. An identification system can be coupled to the fluoroscopic image capture device, the identification system comprising a marker library with first and second image identification data associated with the first and second markers when the image capture device is oriented toward the first major surfaces of the markers, respectively, and third and fourth image identification data associated with the first and second markers when the image capture device is oriented toward the second major surfaces of the markers, respectively. Advantageously, this can allow the identification system to transmit a first identification signal in response to the first marker and a second identification signal in response to the second marker independent of which major surfaces are oriented toward the image capture device. This can be particularly helpful when attempting to identify and determine the pose of one or more interventional or other components of a structural heart system having radiolucent structures by facilitating the interpretation of standard (such as Aruco) or custom libraries of radio-opaque machine-readable markers regardless of the rotational orientation of the components.

### BRIEF DESCRIPTION OF THE DRAWINGS

FIG. 1 illustrates an interventional cardiologist performing a structural heart procedure with a robotic catheter system having a fluidic catheter driver slidably supported by a stand.

FIGS. 2 and 2A are perspective views of components of a robotic catheter system in which a catheter is removably mounted on a driver assembly, in which the driver assembly includes a driver encased in a sterile housing and supported by a stand, and in which the catheter is inserted distally into the patient through an articulated or non-articulated guide sheath.

FIG. 3 schematically illustrates a robotic catheter system and transmission of signals between the components thereof so that input from a user induces a desired articulation.

FIGS. 4A and 4B schematically illustrate a data processing system architecture for a robotic catheter system and transmission of signals between a user interface, a motion controller, and embedded circuitry of a drive assembly.

FIG. 5 is a functional block diagram schematically illustrating software components and data flows of the motion controller of FIGS. 4A and 4B.

FIG. 6 is a functional block diagram schematically illustrating data processing components included in a single-use replaceable catheter and data flows between those components and the data processing components of a reusable driver assembly on which the catheter is mounted.

FIG. 7 is a perspective view of robotic catheter system and a clinical user of that system showing a 3D user input space and a 2D or 3D display space of the system with which the user interacts.

FIG. 8 schematically illustrates images included in the display space of FIG. 7, showing 2D and 3D image components represented in the display space and their associated reference frames as registered using the registration system of the robotic system data processor so that the user environment of the robotic system user interface presents a coherent 3D workspace to the user.

FIGS. 9A-9D illustrate optional image elements to be included in an exemplary hybrid 2D/3D display image of the user interface.

FIGS. 9E and 9E-1 illustrate exemplary echo image planes of the display from a biplane transesophogeal echocardiography (TEE) system, along which planes the user may optionally drive articulation of the robotic catheter using the robotic catheter systems described herein.

FIG. 10 is a block diagram schematically illustrating components and data transmission of a registration module, including a fluoro-based pose module, an echo-based pose module, and a sensor-based pose module.

FIGS. 10A and 10B are screen prints showing output from an image acquisition system calibration module, as used to calibrate camera image data from a camera showing a benchtop model of a therapy site.

FIG. 10C is a screen print showing output from an alignment module as used to align a benchtop model of a therapy site with image data (here from a camera) using automatic recognition and localization of Aruco fiducial markers included in a fiducial marker plate with software developed using the OpenCV software library.

FIG. 10D schematically illustrates output from a pose module which calculates a pose of one or more component of a medical robotic toolset using high-contrast image markers (here using camera image data) and a CAD model of a robotic toolset component.

FIG. 10E is a screen print showing a user interface display in which the robotic data used to control movement of a robotic toolset has been registered with fluoro image data using a fluoro image data alignment module and a planar array of Aurco fiducial markers on a panel that can be positioned between a patient and an interventional treatment table.

FIG. 10F is a screen print showing a user interface display in which the robotic data used to control movement of a robotic catheter of a robotic toolset has been registered with fluoro image data using high-contrast markers on the robotic catheter and a CAD model of the robotic catheter.

FIG. 11A illustrates a user interface display, fluoro and echo image displays, and an image of a user using a robotic catheter system, in which the robotic data and fluoro image data were registered as described herein, during positioning of a virtual catheter in vivo in a right atrium of a beating heart in a porcine model.

FIG. 11B illustrates a user interface display, fluoro and echo image displays, and an image of a user using a robotic catheter system, in which the robotic data and fluoro image data were registered as described herein, during manual insertion of a robotic catheter in which lateral bending of the catheter is controlled robotically so that the distal end of the catheter follows a trajectory based on an axias of the virtual catheter of FIG. 11A in vivo in a right atrium of a beating heart in a porcine model.

FIG. 11C is a screenprint showing in vivo registration of the robotic toolset with the fluoro image data in a right atrium of a beating heart in a porcine model.

FIGS. 12A and 12B are a schematic illustration of tilt angle scanning from a TEE probe and associated TEE X-Plane images, respectively.

FIG. 12C is a 3D echo image space showing schematically how planar echo images from a TEE tilt angle scan can be used to determine an ultrasound-based pose of a toolset component in the 3D echo image space when the component has a lateral bend or eccentric echogenic feature.

FIG. 12D schematically illustrates an alternative TEE X-Plane rotation scan which may be used in place of or in addition to the tilt angle scan of FIG. 12A to determine an ultrasound-based pose of the toolset component(s).

FIGS. 12E and 12F are representative TEE X-Plane image data obtained during a tilt angle scan.

FIG. 13 is a perspective view illustrating a C-arm fluoroscopy image acquisition system and an ultrasound or electromagnetic location sensor.

FIG. 14 is a top view of a panel for positioning between a patient support and a patient, the panel having an exemplary array of Aruco fiducial markers.

FIG. 15 is a perspective view of an exemplary guide sleeve of a robotic toolset, the guide sleeve having an array of Aruco fiducial markers distributed axially and circumferentially with the individual markers bending to a substantially cylindrical arc-segment cross section along the outer profile of the guide sheath.

FIG. 16 is a screen shot from an image-based registration module including both an alignment module that aligns fluoroscopic image data with an internal therapy site using image data from a planar array of fiducial markers, and a fluoroscopy-based pose module that determines a ultrasound-based pose of a guide sheath using image data from a plurality of fiducial markers mounted to the guide sheath together with a CAD model of the guide sheath.

FIGS. 17A-17D illustrate alternative custom 1D and 2D fiducial markers having elongate shapes which may extend along the axis of a catheter or other interventional component to limit cylindrical distortion effects, along with a pattern of such markers on a multi-component toolset.

FIG. 18 illustrates an ultrasound multiplane reconstruction (MPR) display having a plurality of planes and a 3D image which can be registered with a workspace and displayed in the display system described herein.

FIGS. 19A-19C schematically illustrate a guide sheath having a radiolucent body with a pattern of reversible radio-opaque markers that can be read from either of their opposed major surface, along with associated reversible marker library data.

FIG. 20 illustrates exemplary components of an ultrasound data acquisition system.

FIG. 21 illustrates elements included in an echo image data stream and displayed on an exemplary ultrasound display.

FIG. 22 illustrates echo intensity scaling information and characteristics included in an exemplary echo data stream.

FIGS. 23A-23C illustrate elements included in an exemplary echo image data stream, some of which can be captured using optical character recognition technology and/or which relate to electronic steering of the image planes relative to the transducer.

FIG. 24 is a flow chart schematically illustrating an exemplary method for determining pose data regarding an interventional catheter having a bend in response to ultrasound data.

FIG. 25 illustrates physiological movement data extracted from an ultrasound image stream as can be used to understand a method for selecting quiescent data for catheter pose estimation.

FIGS. 26A-26D illustrate 2D echo data, assembly of an associated 3D point cloud, and artifact removal to define a 3D volume.

FIG. 27 is a flow chart showing workflows associated with a graphical user interface (GUI) for echo data capture, registration, and pose data extraction.

FIGS. 28A-28C illustrate aspects of the GUI of FIG. 27.

FIGS. 29A-29C illustrate curve fitting and pose data parameters for determining a pose of an interventional component from echo data.

FIGS. 30A-30D illustrate an exemplary TEE probe having a pattern of machine-readable radio-opaque markers for determining a probe pose.

FIGS. 31A-31D illustrate an exemplary TEE probe having a pattern of machine-readable optical markers for determining a probe pose.

FIG. 32 is a functional block diagram of a multi-thread computer program for registering a plurality of interventional components having parent/child reference coordinate relationships using multi-mode image data; and FIGS. 32A-32E show portions and software modules of that program enlarged for ease of review.

FIG. 33 is a graphical user interface for the computer program of FIG. 32.

### DETAILED DESCRIPTION OF THE INVENTION

The improved devices, systems, and methods for robotic catheters and other systems described herein will find a wide variety of uses. The elongate articulated structures described herein will often be flexible, typically comprising catheters suitable for insertion in a patient body. The structures described herein will often find applications for diagnosing or treating the disease states of or adjacent to the cardiovascular system, the alimentary tract, the airways, the urogenital system, the neurovasculature, and/or other lumen systems of a patient body. Other medical tools making use of the articulation systems described herein may be configured for endoscopic procedures, or even for open surgical procedures, such as for supporting, moving and aligning image capture devices, other sensor systems, or energy delivery tools, for tissue retraction or support, for therapeutic tissue remodeling tools, or the like. Alternative elongate flexible bodies that include the articulation technologies described herein may find applications in industrial applications (such as for electronic device assembly or test equipment, for orienting and positioning image acquisition devices, or the like). Still further elongate articulatable devices embodying the techniques described herein may be configured for use in consumer products, for retail applications, for entertainment, or the like, and wherever it is desirable to provide simple articulated assemblies with one or more (preferably multiple) degrees of freedom without having to resort to complex rigid linkages.

Exemplary systems and structures provided herein may be configured for insertion into the vascular system, the systems typically including a cardiac catheter and supporting a structural heart tool for repairing or replacing a valve of the heart, occluding an ostium or passage, or the like. Other cardiac catheter systems will be configured for diagnosis and/or treatment of congenital defects of the heart, or may comprise electrophysiology catheters configured for diagnosing or inhibiting arrhythmias (optionally by ablating a pattern of tissue bordering or near a heart chamber). Alternative applications may include use in steerable supports of image acquisition devices such as for trans-esophageal echocardiography (TEE), intra-coronary echocardiography (ICE), and other ultrasound techniques, endoscopy, and the like. Still further applications may make use of structures configured as interventional neurovascular therapies that articulate within the vasculature system which circulates blood through the brain, facilitating access for and optionally supporting stroke mitigation devices such as aneurism coils, thrombectomy structures (including those having structures similar to or derived from stents), neurostimulation leads, or the like.

Embodiments described herein may fully or partly rely on pullwires to articulate a catheter or other elongate flexible body. With or without pullwires, alternative embodiments provided herein may use balloon-like structures to effect at least a portion of the articulation of the elongate catheter or other body. The term “articulation balloon” may be used to refer to a component which expands on inflation with a fluid and is arranged so that on expansion the primary effect is to cause articulation of the elongate body. Note that this use of such a structure is contrasted with a conventional interventional balloon whose primary effect on expansion is to cause substantial radially outward expansion from the outer profile of the overall device, for example to dilate or occlude or anchor in a vessel in which the device is located. Independently, articulated medical structures described herein will often have an articulated distal portion and an unarticulated proximal portion, which may significantly simplify initial advancement of the structure into a patient using standard catheterization techniques.

The medical robotic systems described herein will often include an input device, a driver, and a toolset configured for insertion into a patient body. The toolset will often (though will not always) include a guide sheath having a working lumen extending therethrough, and an articulated catheter (sometimes referred to herein as a steerable sleeve) or other robotic manipulator, and a diagnostic or therapeutic tool supported by the articulated catheter, the articulated catheter typically being advanced through the working lumen of the guide sheath so that the tool is at an internal therapy site. The user will typically input commands into the input device, which will generate and transmit corresponding input command signals. The driver will generally provide both power for and articulation movement control over the tool. Hence, somewhat analogous to a motor driver, the driver structures described herein will receive the input command signals from the input device and will output drive signals to the tool-supporting articulated structure so as to effect robotic movement of the tool (such as by inducing movement of one or more laterally deflectable segments of a catheter in multiple degrees of freedom). The drive signals may optionally comprise fluidic commands, such as pressurized pneumatic or hydraulic flows transmitted from the driver to the tool-supporting catheter along a plurality of fluid channels. Optionally, the drive signals may comprise mechanical, electromechanical, electromagnetic, optical, or other signals, with or without fluidic drive signals. Many of the systems described herein inducement movement using fluid pressure. Unlike many robotic systems, the robotic tool supporting structure will often (though not always) have a passively flexible portion between the articulated feature (typically disposed along a distal portion of a catheter or other tool manipulator) and the driver (typically coupled to a proximal end of the catheter or tool manipulator). The system may be driven while sufficient environmental forces are imposed against the tool or catheter to impose one or more bend along this passive proximal portion, the system often being configured for use with the bend(s) resiliently deflecting an axis of the catheter or other tool manipulator by 10 degrees or more, more than 20 degrees, or even more than 45 degrees.

The catheter bodies (and many of the other elongate flexible bodies that benefit from the inventions described herein) will often be described herein as having or defining an axis, such that the axis extends along the elongate length of the body. As the bodies are flexible, the local orientation of this axis may vary along the length of the body, and while the axis will often be a central axis defined at or near a center of a cross-section of the body, eccentric axes near an outer surface of the body might also be used. It should be understood, for example, that an elongate structure that extends “along an axis” may have its longest dimension extending in an orientation that has a significant axial component, but the length of that structure need not be precisely parallel to the axis. Similarly, an elongate structure that extends “primarily along the axis” and the like will generally have a length that extends along an orientation that has a greater axial component than components in other orientations orthogonal to the axis. Other orientations may be defined relative to the axis of the body, including orientations that are transvers to the axis (which will encompass orientation that generally extend across the axis, but need not be orthogonal to the axis), orientations that are lateral to the axis (which will encompass orientations that have a significant radial component relative to the axis), orientations that are circumferential relative to the axis (which will encompass orientations that extend around the axis), and the like. The orientations of surfaces may be described herein by reference to the normal of the surface extending away from the structure underlying the surface. As an example, in a simple, solid cylindrical body that has an axis that extends from a proximal end of the body to the distal end of the body, the distal-most end of the body may be described as being distally oriented, the proximal end may be described as being proximally oriented, and the curved outer surface of the cylinder between the proximal and distal ends may be described as being radially oriented. As another example, an elongate helical structure extending axially around the above cylindrical body, with the helical structure comprising a wire with a square cross section wrapped around the cylinder at a 20 degree angle, might be described herein as having two opposed axial surfaces (with one being primarily proximally oriented, one being primarily distally oriented). The outermost surface of that wire might be described as being oriented exactly radially outwardly, while the opposed inner surface of the wire might be described as being oriented radially inwardly, and so forth.

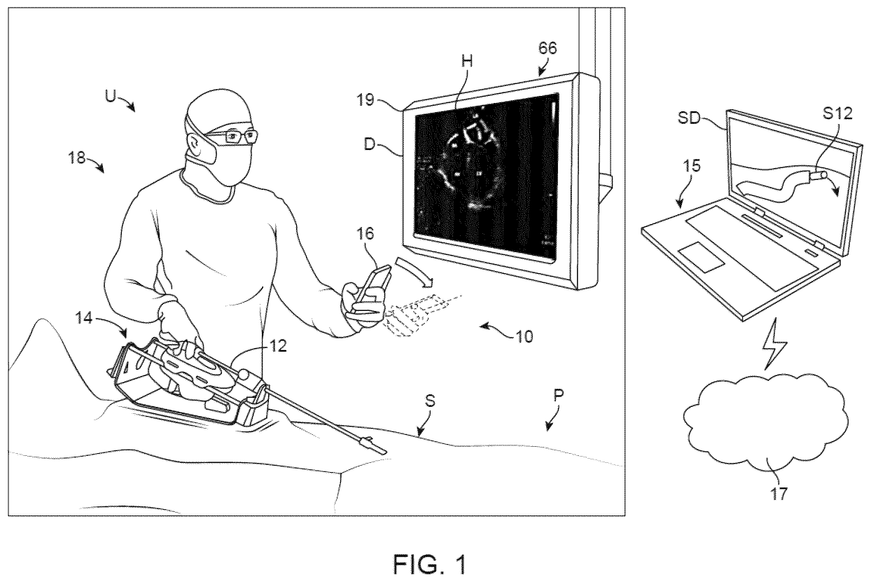

Referring first to FIG. 1, a system user U, such as an interventional cardiologist, uses a robotic catheter system 10 to perform a procedure in a heart H of a patient P. System 10 generally includes an articulated catheter 12, a driver assembly 14, and an input device 16. User U controls the position and orientation of a therapeutic or diagnostic tool mounted on a distal end of catheter 12 by entering movement commands into input 16, and optionally by axially moving the catheter relative to a stand of the driver assembly, while viewing an image of the distal end of the catheter and the surrounding tissue in a display D.

During use, catheter 12 extends distally from driver system 14 through a vascular access site S, optionally (though not necessarily) using an introducer sheath. A sterile field 18 encompasses access site S, catheter 12, and some or all of an outer surface of driver assembly 14. Driver assembly 14 will generally include components that power automated movement of the distal end of catheter 12 within patient P, with at least a portion of the power often being generated and modulated using hydraulic or pneumatic fluid flow. To facilitate movement of a catheter-mounted therapeutic tool per the commands of user U, system 10 will typically include data processing circuitry, often including a processor within the driver assembly. Regarding that processor and the other data processing components of system 10, a wide variety of data processing architectures may be employed. The processor, associated pressure and/or position sensors of the driver assembly, and data input device 16, optionally together with any additional general purpose or proprietary computing device (such as a desktop PC, notebook PC, tablet, server, remote computing or interface device, or the like) will generally include a combination of data processing hardware and software, with the hardware including an input, an output (such as a sound generator, indicator lights, printer, and/or an image display), and one or more processor board(s). These components are included in a processor system capable of performing the transformations, kinematic analysis, and matrix processing functionality associated with generating the valve commands, along with the appropriate connectors, conductors, wireless telemetry, and the like. The processing capabilities may be centralized in a single processor board, or may be distributed among various components so that smaller volumes of higher-level data can be transmitted. The processor(s) will often include one or more memory or other form of volatile or non-volatile storage media, and the functionality used to perform the methods described herein will often include software or firmware embodied therein. The software will typically comprise machine-readable programming code or instructions embodied in non-volatile media and may be arranged in a wide variety of alternative code architectures, varying from a single monolithic code running on a single processor to a large number of specialized subroutines, classes, or objects being run in parallel on a number of separate processor sub-units.

Referring still to FIG. 1, along with display D, a simulation display SD may present an image of an articulated portion of a simulated or virtual catheter S12 with a receptacle for supporting a simulated therapeutic or diagnostic tool. The simulated image shown on the simulation display SD may optionally include a tissue image based on pre-treatment imaging, intra-treatment imaging, and/or a simplified virtual tissue model, or the virtual catheter may be displayed without tissue. Simulation display SD may have or be included in an associated computer 15, and the computer will preferably be couplable with a network and/or a cloud 17 so as to facilitate updating of the system, uploading of treatment and/or simulation data for use in data analytics, and the like. Computer 15 may have a wireless, wired, or optical connection with input device 16, a processor of driver assembly 14, display D, and/or cloud 17, with suitable wireless connections comprising a Bluetooth™ connection, a WiFi connection, or the like. Preferably, an orientation and other characteristics of simulated catheter S12 may be controlled by the user U via input device 16 or another input device of computer 15, and/or by software of the computer so as to present the simulated catheter to the user with an orientation corresponding to the orientation of the actual catheter as sensed by a remote imaging system (typically a fluoroscopic imaging system, an ultra-sound imaging system, a magnetic resonance imaging system (MRI), or the like) incorporating display D and an image capture device 19. Optionally, computer 15 may superimpose an image of simulated catheter S12 on the tissue image shown by display D (instead of or in addition to displaying the simulated catheter on simulation display SD), preferably with the image of the simulated catheter being registered with the image of the tissue and/or with an image of the actual catheter structure in the therapy or surgical site. Still other alternatives may be provided, including presenting a simulation window showing simulated catheter SD on display D, including the simulation data processing capabilities of computer 15 in a processor of driver assembly 14 and/or input device 16 (with the input device optionally taking the form of a tablet) that can be supported by or near driver assembly 14, incorporating the input device, computer, and one or both of displays D, SD into a workstation near the patient, shielded from the imaging system, and/or remote from the patient, or the like.

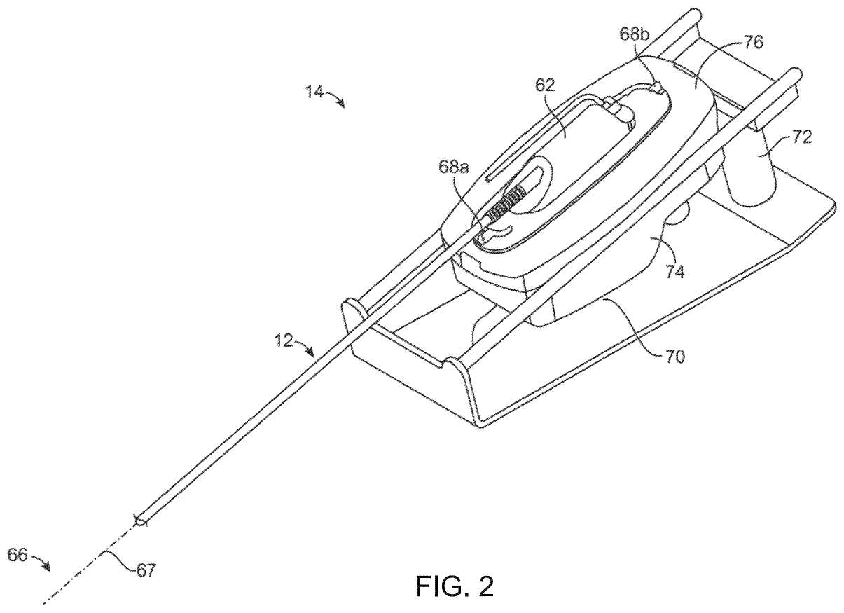

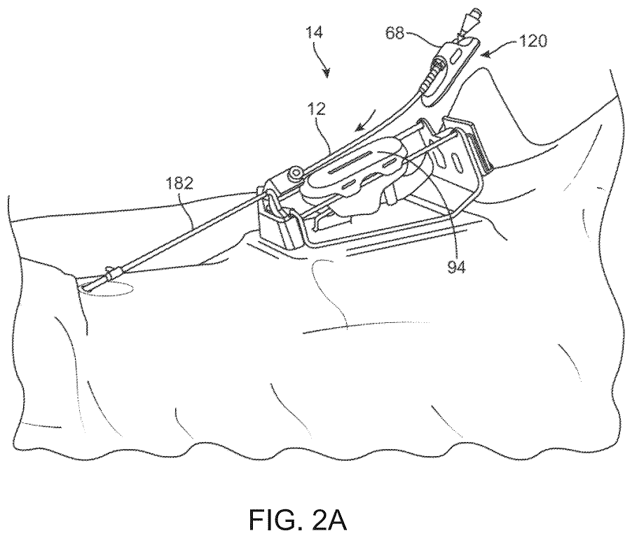

Referring now to FIG. 2, catheter 12 is removably mounted on exemplary driver assembly 14 for use. Catheter 12 has a proximal portion 22 and a distal portion 24 with an axis 26 extending therebetween. A proximal housing 28 of catheter 12 has an interface 30 that sealingly couples with an interface 32 of a driver 34 included in driver assembly 14 so that fluid drive channels of the driver are individually sealed to fluid channels of the catheter housing, allowing separate pressures to be applied to control the various degrees of freedom of the catheter. Driver 34 is contained within a sterile housing 36 of driver assembly 14. Driver assembly 14 also includes a support 38 or stand with rails extending along the axis 26 of the catheter, and the sterile housing, driver, and proximal housing of the catheter are movably supported by the rails so that the axial position of the catheter and the associated catheter drive components can move along the axis under either manual control or with powered robotic movement. Details of the sterile housing, the housing/driver interface, and the support are described in PCT Patent Publication No. WO 2019/195841, assigned to the assignee of the subject application and filed on Apr. 8, 2019, the full disclosure of which is incorporated herein by reference.

Referring now to FIG. 2A, a guide sheath 182 is introduced into and advanced within the vasculature of the patient, optionally through an introducer sheath (though no introducer sheath may be used in alternate embodiments). Guide sheath 182 may optionally have a single pull-wire for articulation of a distal portion of the guide sheath, similar to the guide catheter used with the MitraClip™ mitral valve therapy system as commercially available from Abbott. Alternatively, the guide sheath may be an unarticulated tubular structure that can be held straight by the guidewire or a dilator extending within the lumen along the bend, or use of the guide sheath may be avoided. Regardless, when used the guide sheath will often be advanced manually by the user toward a surgical site over a guidewire, with the guide sheath often being advanced up the inferior vena cava (IVC) to the right atrium, and optionally through the septum into the left atrium. Driver assembly 14 may be placed on a support surface, and the driver assembly may be slid along the support surface roughly into alignment with the guide sheath 182. A proximal housing of guide sheath 182 can be releasably affixed to a catheter support of stand 72, with the support typically allowing rotation of the guide sheath prior to full affixation (such as by tightening a clamp of the support). Catheter 12 can be advanced distally through the guide sheath 182, with the user manually manipulating the catheter by grasping the catheter body and/or proximal housing 68. Note that the manipulation and advancement of the access wire, guide catheter, and catheter to this point may be performed manually so as to provide the user with the full benefit of tactile feedback and the like. As the distal end of catheter 12 extends near, to, or from a distal end of the guide sheath into the therapy area adjacent the target tissue (such as into the right or left atrium) by a desired amount, the user can manually bring the catheter interface 120 down into engagement with the driver interface 94, preferably latching the catheter to the driver through the sterile junction.

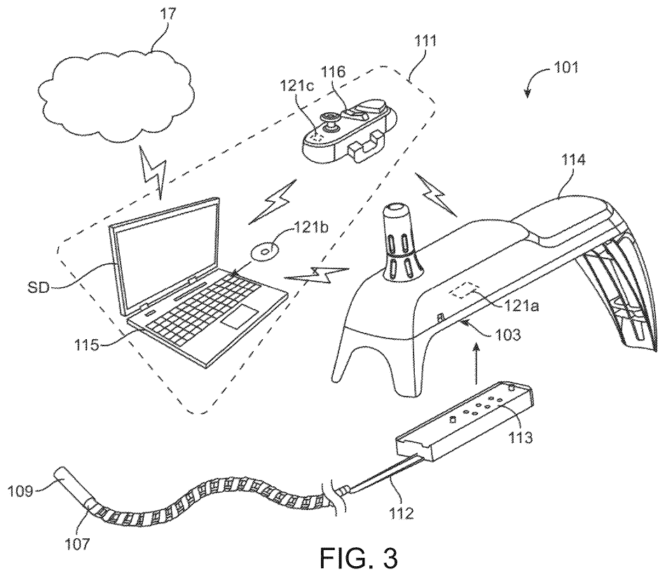

Referring now to FIG. 3, components of a networked system 101 that can be used for simulation, training, pre-treatment planning, and or treatment of a patent are schematically illustrated. Some or all of the components of system 101 may be used in addition to or instead of the clinical components of the system shown in FIG. 1. System 101 may optionally include an alternative catheter 112 and an alternative driver assembly 114, with the alternative catheter comprising a real and/or virtual catheter and the driver assembly comprising a real and/or virtual driver 114. Alternative catheter 112 can be replaceably coupled with alternative driver assembly 114. When system 101 is used for driving an actual catheter, the coupling may be performed using a quick-release engagement between an interface 113 on a proximal housing of the catheter and a catheter receptacle 103 of the driver assembly. An elongate body 105 of catheter 112 has a proximal/distal axis as described above and a distal receptacle 107 that is configured to support a therapeutic or diagnostic tool 109 such as a structural heart tool for repairing or replacing a valve of a heart. Alternative drive assembly 114 may be wireless coupled to a computer 115 and/or an input device 116. Software modules embodying machine-readable code for implementing the methods described therein will often comprise software modules, and the modules will optionally be embodied at least in-part in a non-volatile memory of alternative drive assembly 121a or an associated computer, but some or all of the simulation modules will preferably be embodied as software in non-volatile memories 121b, 121c of a computer 115 and/or input device 116, respectively.

Computer 115 preferably comprises a proprietary or off-the-shelf notebook or desktop computer that can be coupled to cloud 17, optionally via an intranet, the internet, an ethernet, or the like, typically using a wireless router or a cable coupling the simulation computer to a server. Cloud 17 will preferably provide data communication between simulation computer 115 and a remote server, with the remote server also being in communication with a processor of other computers 115 and/or one or more clinical drive assemblies 14. Computer 115 may also comprise code with a virtual 3D workspace, the workspace optionally being generated using a proprietary or commercially available 3D development engine that can also be used for developing games and the like, such as Unity™ as commercialized by Unity Technologies. Suitable off-the-shelf computers may include any of a variety of operating systems (such as Windows from Microsoft, OS from Apple, Linex, or the like), along with a variety of additional proprietary and commercially available apps and programs.

Input device 116 may comprise an off-the-shelf input device having a sensor system for measuring input commands in at least two degrees of freedom, preferably in 3 or more degrees of freedom, and in some cases 5, 6, or more degrees of freedom. Suitable off-the-shelf input devices include a mouse (optionally with a scroll wheel or the like to facilitate input in a 3rd degree of freedom), a tablet or phone having an X-Y touch screen (optionally with AR capabilities such as being compliant with ARCore from Google, ARKit from Apple, or the like to facilitate input of translation and/or rotation, a gamepad, a 3D mouse, a 3D stylus, or the like. Proprietary code may be loaded on the input device (particularly when a phone, tablet, or other device having a touchscreen is used), with such input device code presenting menu options for inputting additional commands and changing modes of operation of the simulation or clinical robotic system.

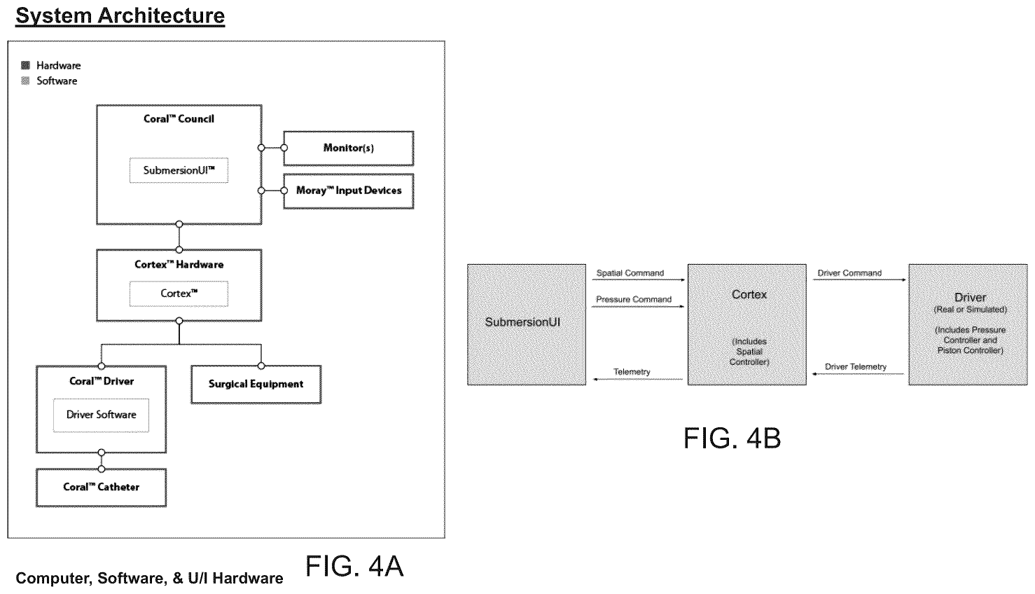

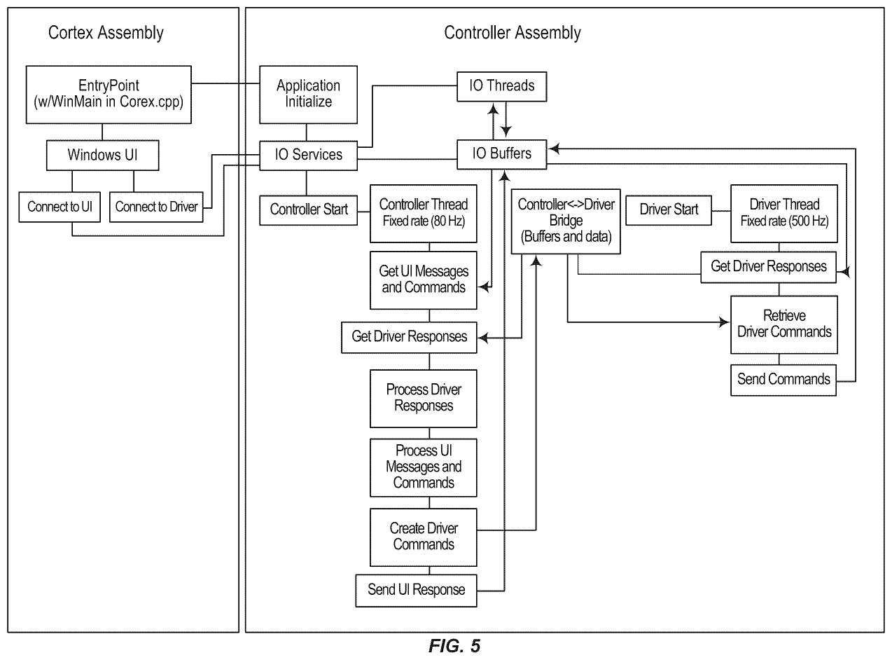

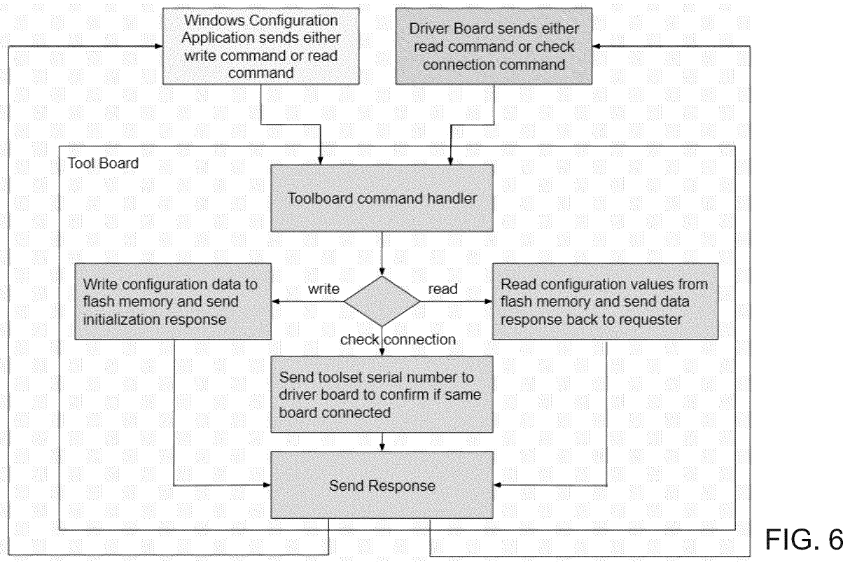

Referring now to FIGS. 4A and 4B, a data processing system architecture for a robotic catheter system employs signals transmitted between a user interface, a motion controller, and embedded circuitry of a drive assembly. As can be seen in a functional block diagram of FIG. 5, software components and data flows into and out of the motion controller of FIGS. 4A and 4B are used by the motion controller to effect movement of, for example, a distal end of the steerable sleeve toward a position and orientation that aligns with the input from the system user. The functional block diagram of FIG. 6 schematically illustrates data processing components included in each single-use replaceable catheter and data flows between those components and the data processing components of the reusable driver assembly on which the catheter is mounted. The motion controller makes use to this catheter data so that the kinematics of different catheters do not fundamentally alter the core user interaction when, for example, positioning a tool carried by a catheter within the robotic workspace.

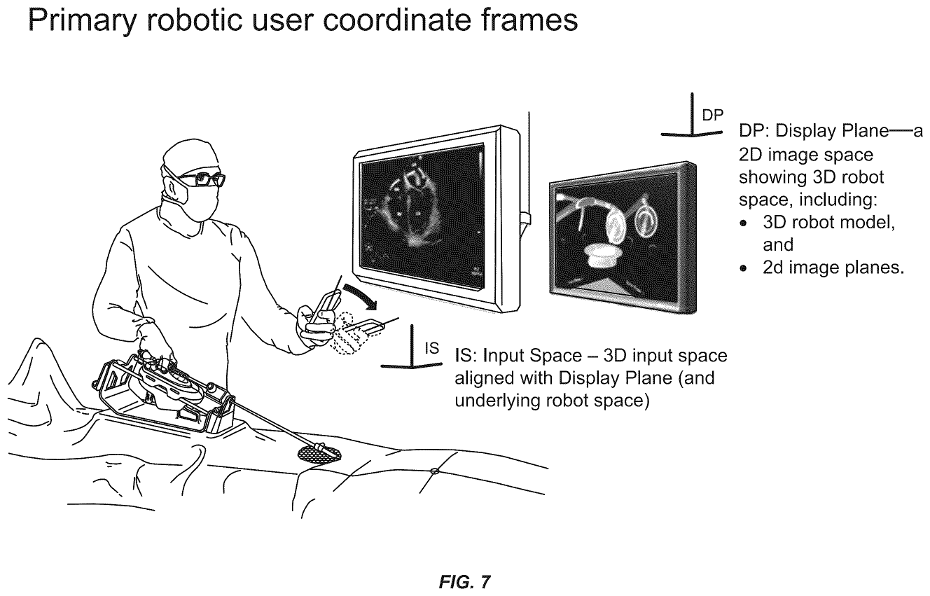

Referring now to FIG. 7, a perspective view of the robotic catheter system in the cathlab shows a clinical user of that system interacting with a 3D user input space by moving the input device therein, and with a 2D display space of the system by viewing the toolset and tissues in the images shown on the planar display. Note that in alternative embodiments the user may view a 3D display space using any of a wide variety of 3D display systems developed for virtual reality (VR), augmented reality (AR), 3D medical image display, or the like, including stereoscopic glasses with an alternating display screen, VR or AR headsets or glasses, or the like.

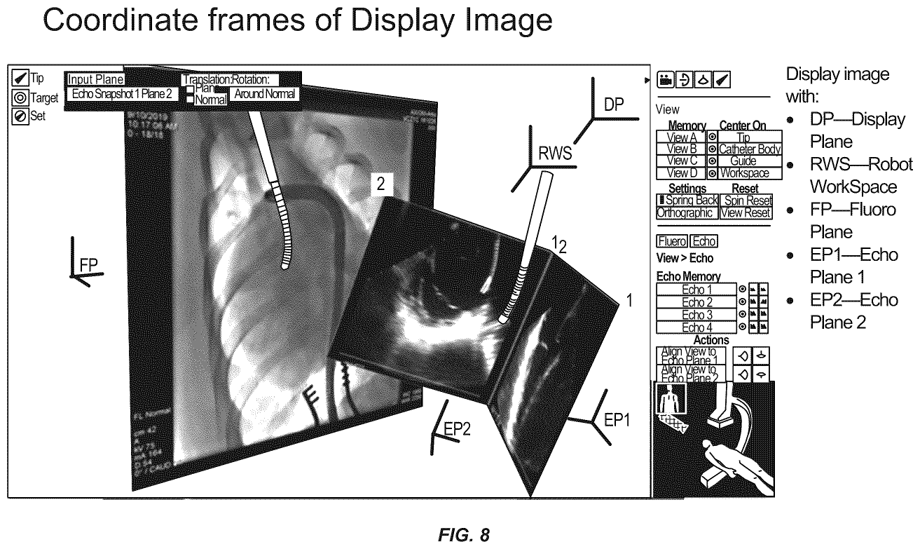

Referring now to FIG. 8, images included in the display space of FIG. 7, may include 2D and 3D image components, with these image components often representing both 3D robotic data and 2D or 3D image data. The image data will often comprise in situ images, optionally comprising live image streams or recorded video or still images from the off-the-shelf image acquisition devices used for image guided therapies, such as planar fluoroscopy images, 2D or 3D ultrasound images, and the like. The robotic data may comprise 3D models, and may be shown as 3D objects in the display and/or may be projected onto the 2D image planes of the fluoro and echo images. Regardless, appropriate positioning of the image and robotic data, as they are represented in the display space helps the user environment of the robotic system user interface to presents a coherent 3D workspace to the user. Toward that end, proper alignment of the reference frames associated with the robotic data, echo data, and fluoro data will generally be provided by the registration system of the robotic system data processor.

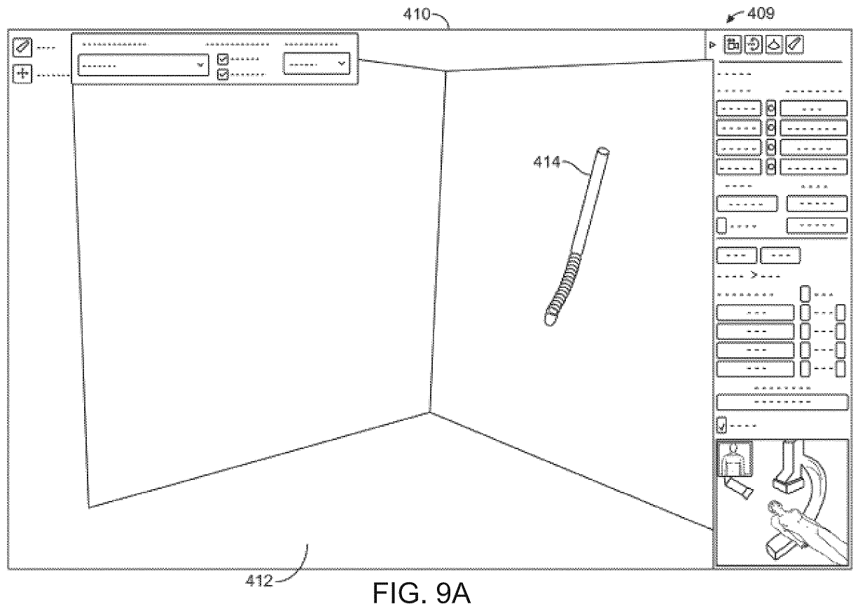

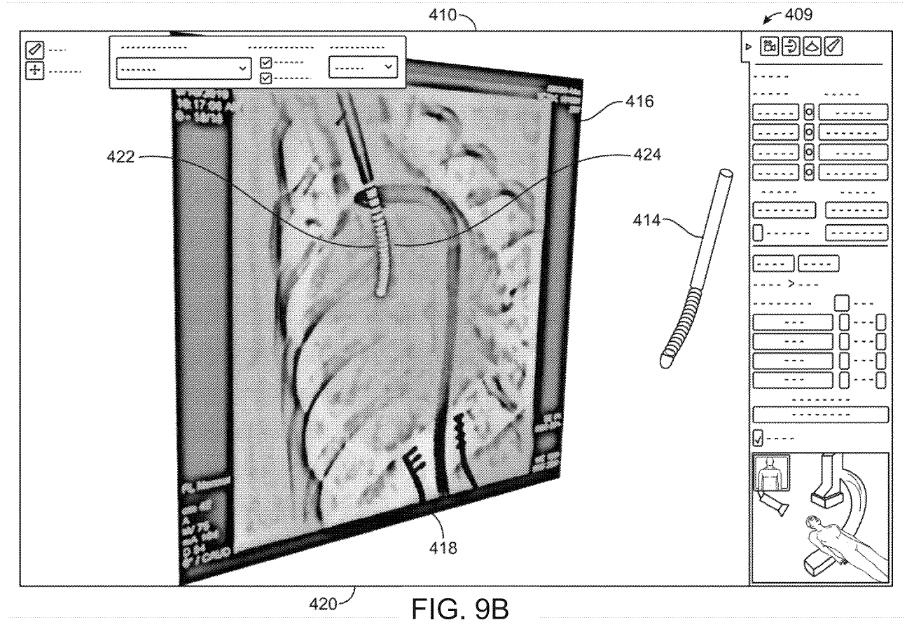

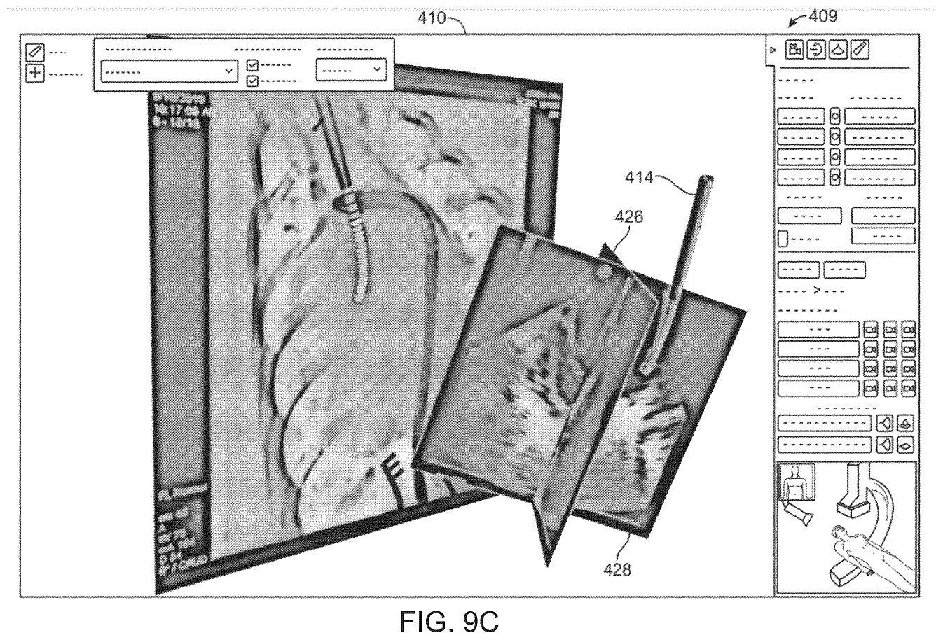

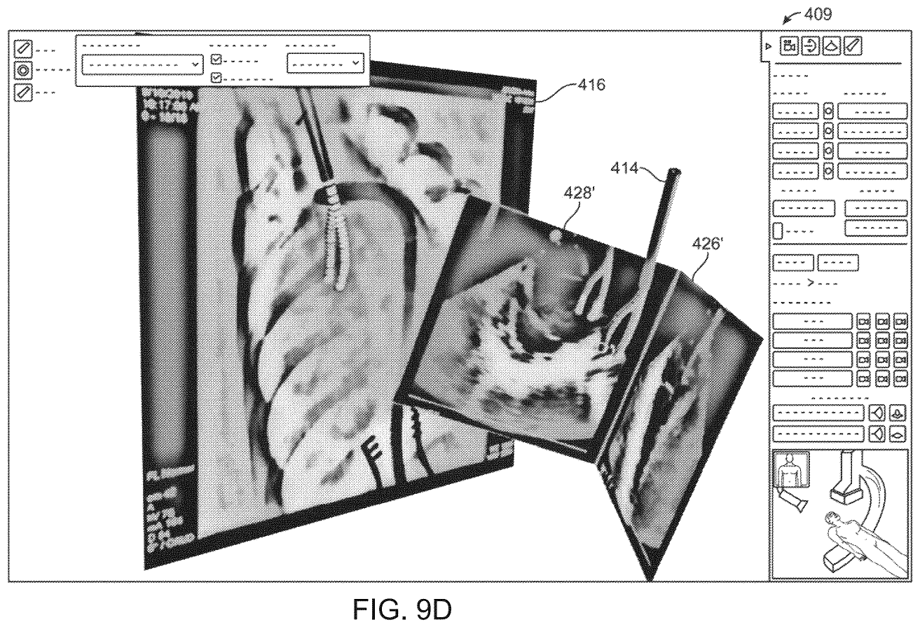

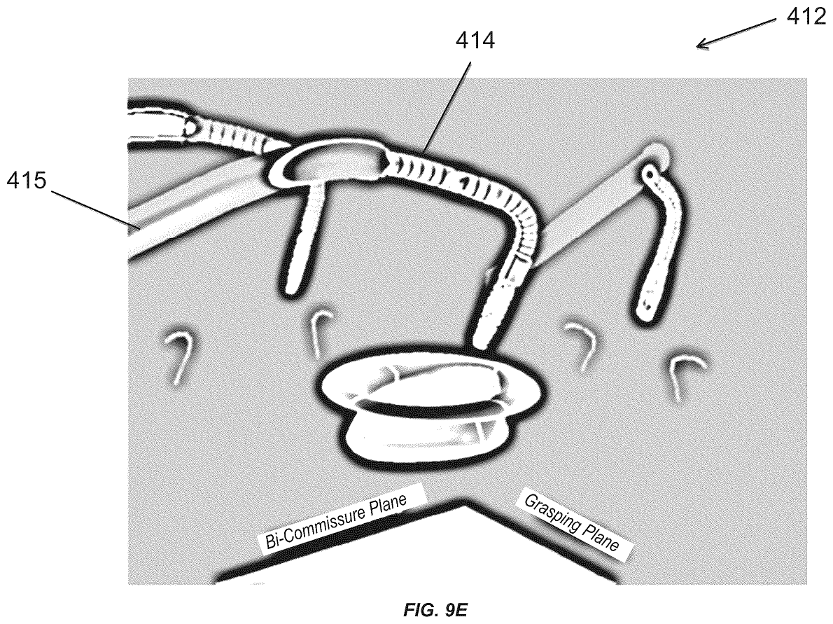

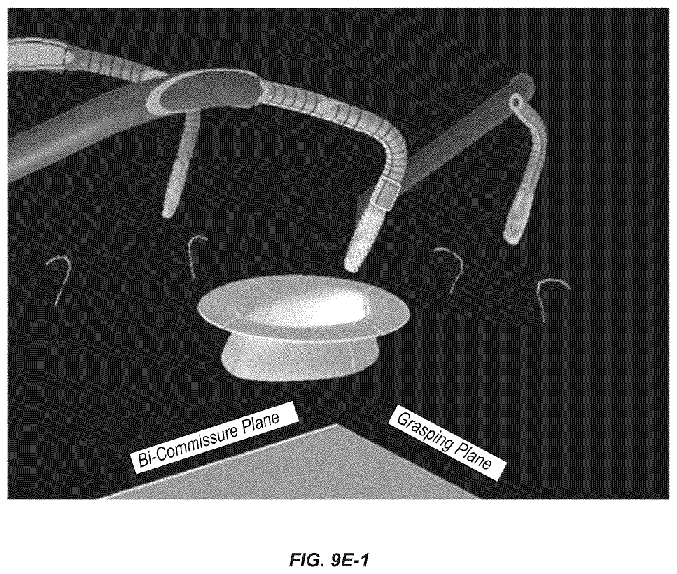

Referring now to FIGS. 9A-9E-1, image data from off-the-shelf or proprietary image acquisition systems and virtual robotic data may be included in a hybrid 2D/3D image to be presented to a system user on a display 410, with the image components generally being presented in a virtual 3D workspace 412 that corresponds to an actual therapeutic workspace within a patient body. A 3D image of a catheter 414 defines a pose in workspace 412, with the shape of the catheter often being determined in response to pressure and/or other drive signals of the robotic system, and optionally in response to imaging, electromagnetic, or other sensor signals so that the catheter image corresponds to an actual shape of an actual catheter. Similarly, a position and orientation of the 3D catheter image 414 in 3D workspace 412 corresponds to an actual catheter based on drive and/or feedback signals.

Referring to FIGS. 9A-9D, additional elements may optionally be included in image 409 such as a 2D fluoroscopic image 416, the fluro image having an image plane 418 which may be shown at an offset angle relative to a display plane 420 of image 410 so that the fluoro image and the 3D virtual image of catheter 414 correspond in the 3D workspace. Fluoro image 416 may include an actual image 422 of an actual catheter in the patient, as well as images of adjacent tissues and structures (including surgical tools). A virtual 2D image 424 of 3D virtual catheter 414 may be projected onto the fluoro image. As seen in FIG. 9C, transverse or X-plane planar echo images 426, 428 may similarly be included in hybrid image 409 at the appropriate angles and locations relative to the virtual 3D catheter 414, with 2D virtual images of the virtual catheter optionally being projected thereon. However, as shown in FIG. 9D, it will often be advantageous to offset the echo image planes from the virtual catheter to generate associated offset echo images 426′, 428′ that can more easily be seen and referenced while driving the virtual or actual catheter. The planar fluoro and echo images within the hybrid image 409 will preferably comprise streaming live actual video obtained from the patient when the catheter is being driven. As can be understood with reference to FIGS. 9E and 9E-1, the virtual or actual catheter may optionally be driven along a selected image plane (often a selected echo image plane showing a target tissue) by constraining movement of the catheter to that plane, thereby helping maintain desired visibility of the catheter to echo imaging and/or alignment of the catheter in other degrees of freedom. Catheter 414, sometimes referred to as a steerable sleeve, extends through a lumen of a guide sheath 415 and distal of the distal end of the guide, with the guide typically being laterally flexible for insertion to the workspace but somewhat stiffer than the catheter so as to act as a robotic base during robotic articulation of the catheter within the workspace.

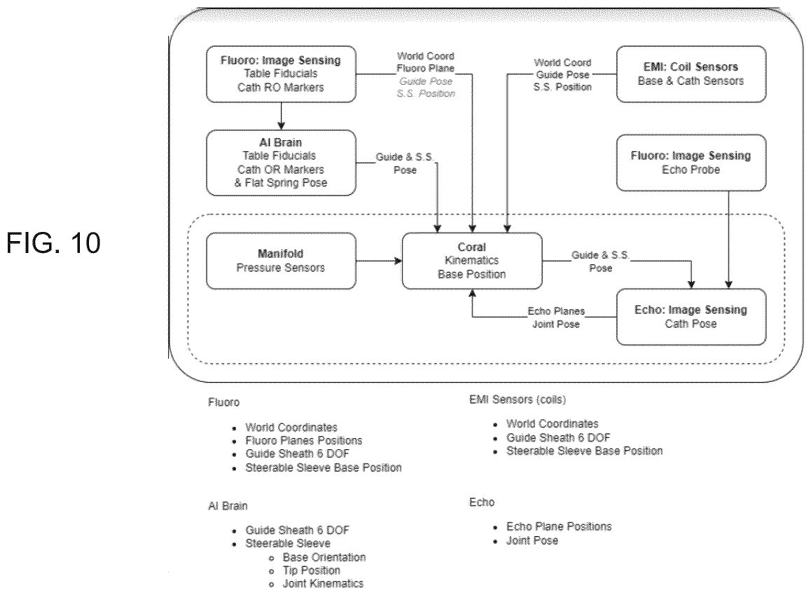

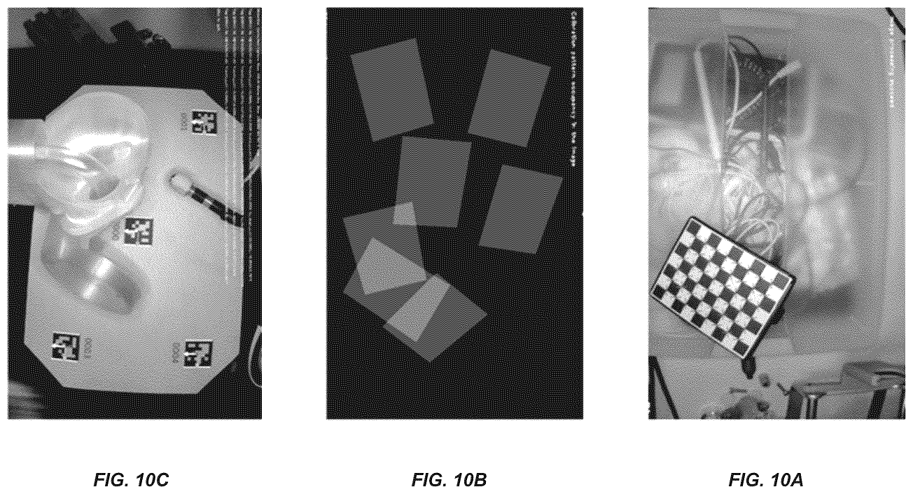

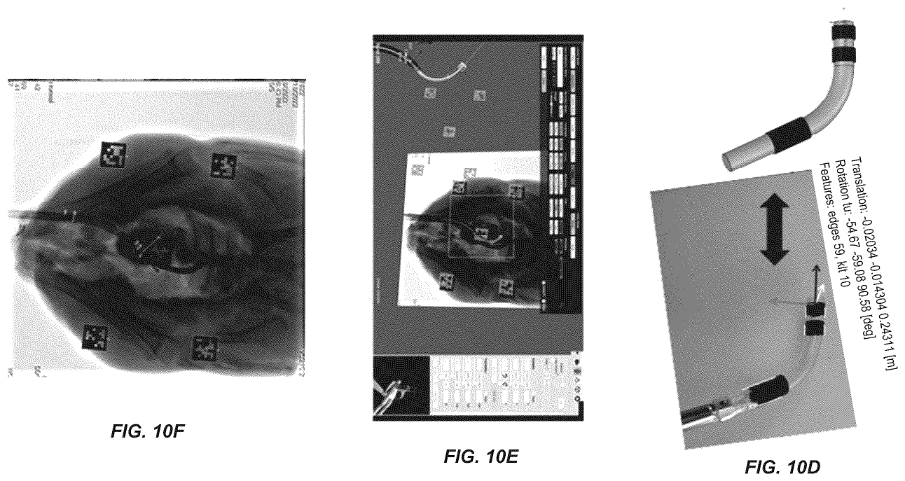

Referring now to FIG. 10, a block diagram schematically illustrating components and data transmission of a registration module generally includes a fluoro-based pose module, an echo-based pose module, and a sensor-based pose module. One, two, or three of the pose-determining modules will provide data to a robot movement control module, which may also provide robotic data regarding the pose of the articulated toolset such as pressure or fluid volume for the fluid-drive systems described herein. A fluoro image data-based pose system includes a C-arm and a fluoro image data processing module that determines an alignment between an internal therapy site and fluoro image data, and optionally a fluoro-image-based pose of one or more components of a robotic toolset in the robotic workspace, An echo image data-based system may include a TEE probe and an echo image data processing module that determines an echo-data-based pose of one or more toolset components in an echo workspace. Registration of the echo-based pose and the fluoro-based posed so as to register the echo workspace and echo image space (including the echo planes) with the robotic workspace of the 3D user environment may take place in the overall motion control module, or in a separate registration module which sends data to and receives data from the robotic motion control module. An electromagnetic (EMI) pose system (which includes an EMI sensor and a sensor data processing module) may similarly provide pose data to such an integrated or separate registration module. Note that the pose modules need not be separate. For example, data from the fluoro pose module or EMI pose module may be used by the echo pose module. Each of the pose modules may make use of data from the robotic motion control module, such as a profile or robot-data based pose of the toolset.

Referring to FIGS. 7-10, the robotic systems described herein can diagnose or treat a patient by receiving fluoroscopic image data with a data processor of the medical robotic system. The fluoroscopic image data preferably encompasses a portion of a toolset of the medical robotic system within a therapy site of the patient, with the portion typically including a distal portion of a guide sheath and/or at least part of the articulatable distal portion of the steerable sleeve. Ultrasound image data is also received with the data processor, the ultrasound image data encompassing the portion of the toolset and a target tissue of the patient within an ultrasound image field. Note that when the target tissue comprises a soft tissue such as a valve tissue of the heart, the target tissue may be more easily visible with the echo data than with the fluoro data. The data processor can determine, in response to the fluoroscopic image data, an alignment of the toolset with the therapy site, often using one or more fiducial markers that are represented in the fluoro data. The data processor can determine, in response to the ultrasound image data, an ultrasound-based pose of the toolset within the ultrasound image field. The data processor can transmit, based in-part on a desired movement of the toolset relative to the target tissue in the ultrasound image field as input by the user, and in-part on the alignment, and in-part on the ultrasound-based pose, a command for articulating the tool. To generate that command, the processor will often calculate, based on the ultrasound-based pose and the alignment, a registration of the ultrasound image field with the therapy site, with the registration optionally comprising a transformation between the ultrasound image field and the 3D robotic workspace.

Referring now to FIGS. 10-11C and 14-16, the fluoroscopic data-based pose module may help generate the desired alignment between the reference frames associated with the fluoroscopic and robotic data by calibrating fluoroscopic image data generated by a fluoroscopic image acquisition system. A processor of the medical robotic system can determine, in response to the calibrated fluoroscopic image data, an alignment of the fluoroscopic image data with a therapy site by imaging the therapy site and a plurality of fiducial markers using the fluoroscopic image acquisition system. The fluoro system may also capture toolset image data, the toolset image data encompassing a portion of a toolset in the therapy site. The processor can calculate, in response to the captured toolset image data and the determined alignment, a pose of the toolset in the therapy site, and can use that pose to drive movement of the toolset in the therapy site using the pose.

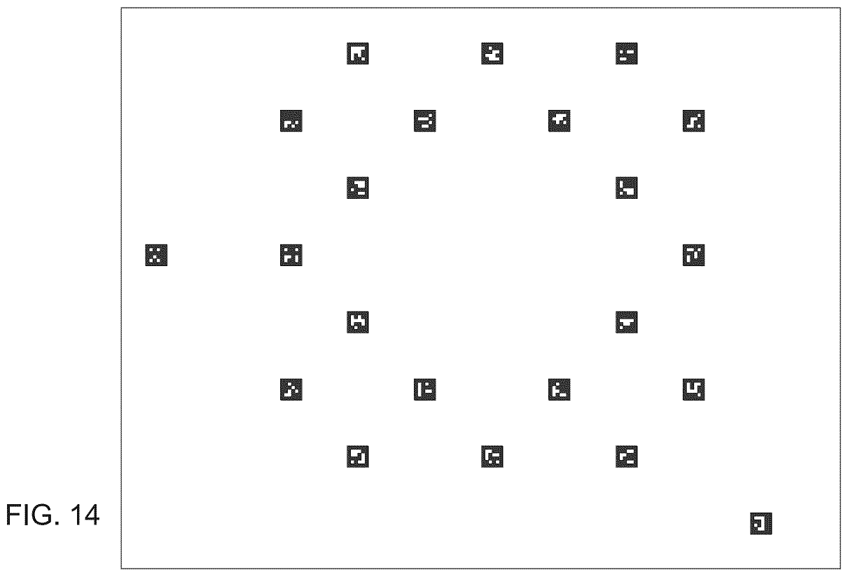

FIGS. 10A and 10B show screen prints of output from an image acquisition system calibration module, as used to calibrate camera image data from a camera showing a benchtop model of a therapy site. The calibration module may make use of an OpenCV software library, and the calibration images may encompass a known calibration target such as a checkerboard or Charuco pattern. For visibility under fluoroscopy, such a pattern may be formed from a high-visibility material, such as by laser cutting the calibration target from tantalum or the like. FIG. 10C is a screen print showing output from an alignment module as used to align a benchtop model of a therapy site with image data (here from a camera). High-contrast fiducial markers for fluoro imaging may again be laser cut from tantalum. Automatic recognition and localization of the markers may be facilitated by the use of markers embodying machine-readable 1D or 2D codes that have been developed for camera-imaging, such as Aruco markers or the like. These fiducial markers may be included in a fiducial marker plate such as that shown in FIG. 14. The fluoro-based pose module code for processing fluoro image including some or all of the markers from such a plate may again use the OpenCV software library.

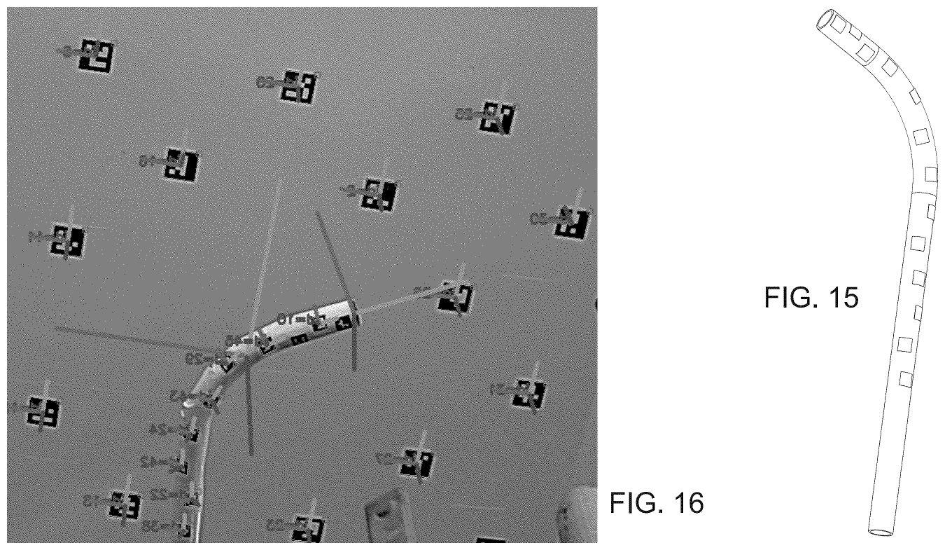

Referring now to FIG. 10D, output from a pose module which calculates a pose of one or more component of a medical robotic toolset using high-contrast image markers (here using camera image data) and a CAD model of a robotic toolset component corresponding to the imaged component with its markers can be seen. As can be understood with reference to FIGS. 15 and 16, machine-readable fiducial markers mounted to the imaged component can facilitate automatic identification, localization, and tracking of the robotic toolset in the image field. Note that not all of the fiducial markers may be identified in a single image, so that integrating the marker localization data over a time period may improve fluoro-based pose estimation. FIG. 10E is a screen print showing a user interface display in which the robotic data used to control movement of a robotic toolset has been registered with fluoro image data using a fluoro image data alignment module and a planar array of Aurco fiducial markers on a panel that can be positioned between a patient and an interventional treatment table. FIG. 10F is a screen print showing a user interface display in which the robotic data used to control movement of a robotic catheter of a robotic toolset has been registered with fluoro image data using high-contrast markers on the robotic catheter and a CAD model of the robotic catheter.

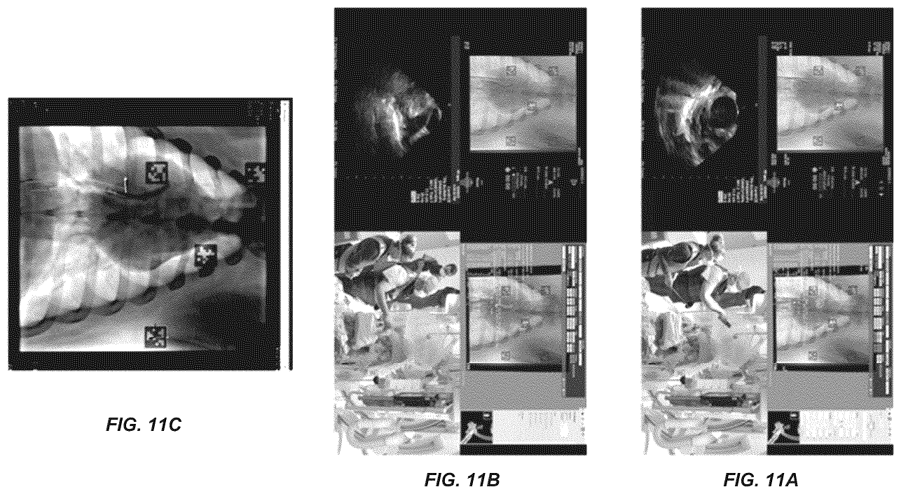

FIG. 11A illustrates a user interface display, fluoro and echo image displays, and an image of a user using a robotic catheter system, in which the robotic data and fluoro image data were registered as described herein, during positioning of a virtual catheter in vivo in a right atrium of a beating heart in a porcine model.

FIG. 11B illustrates a user interface display, fluoro and echo image displays, and an image of a user using a robotic catheter system, in which the robotic data and fluoro image data were registered as described herein, during manual insertion of a robotic catheter in which lateral bending of the catheter is controlled robotically so that the distal end of the catheter follows a trajectory based on an axis of the virtual catheter of FIG. 11A in vivo in a right atrium of a beating heart in a porcine model.

FIG. 11C is a screenprint showing in vivo registration of the robotic toolset with the fluoro image data in a right atrium of a beating heart in a porcine model.

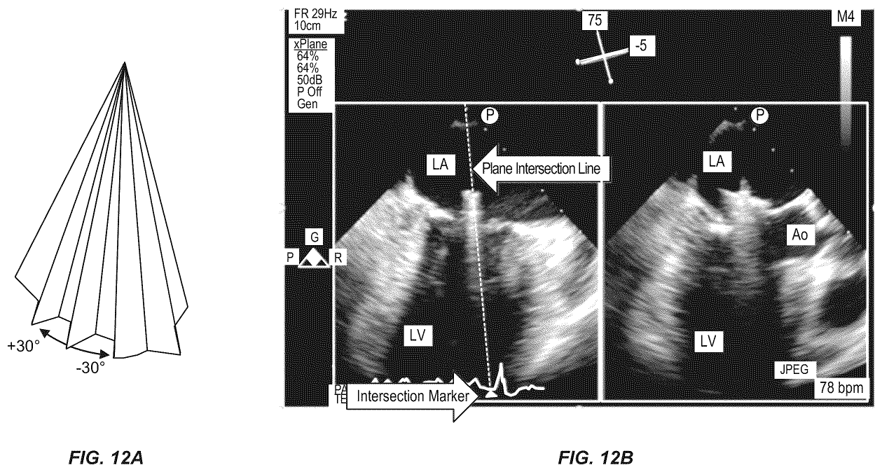

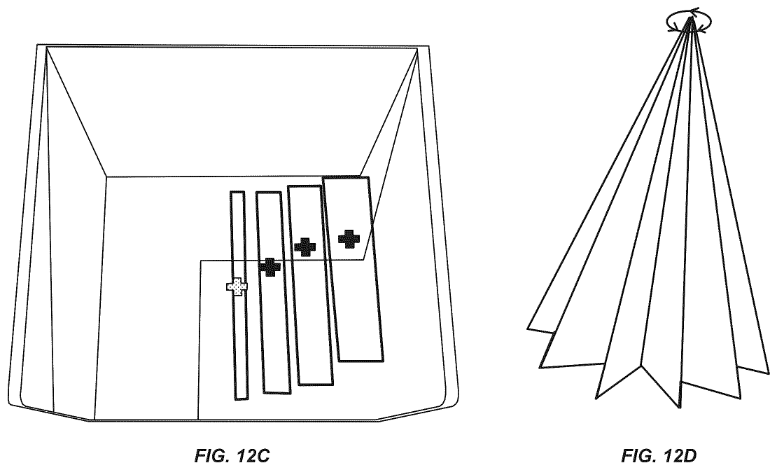

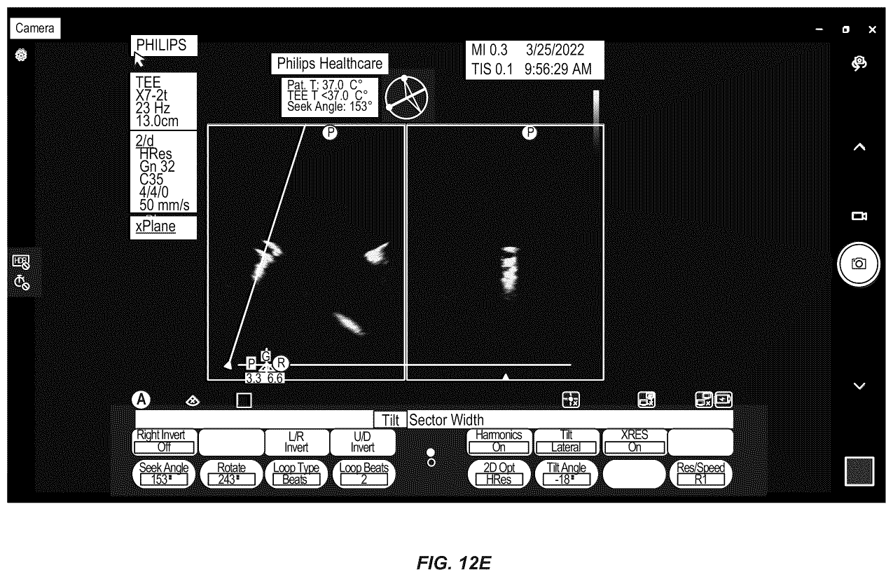

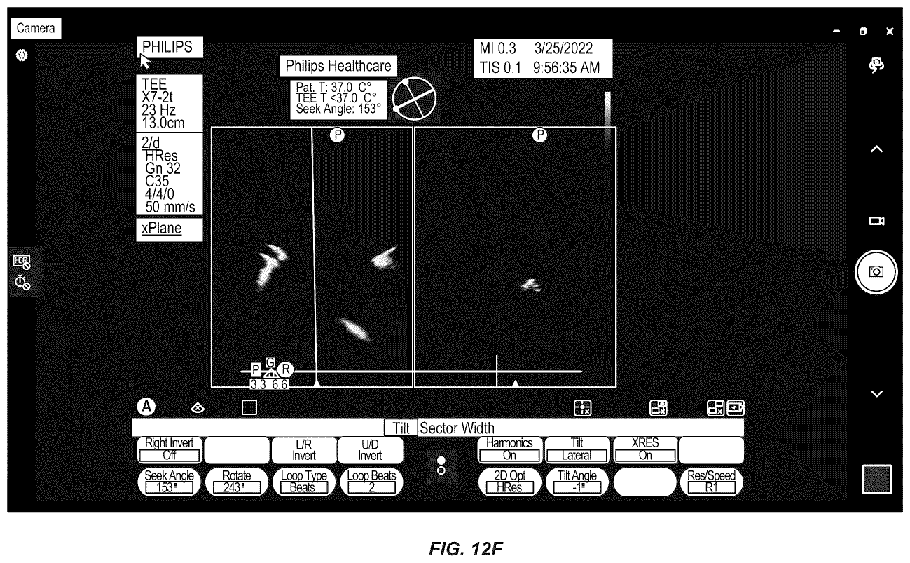

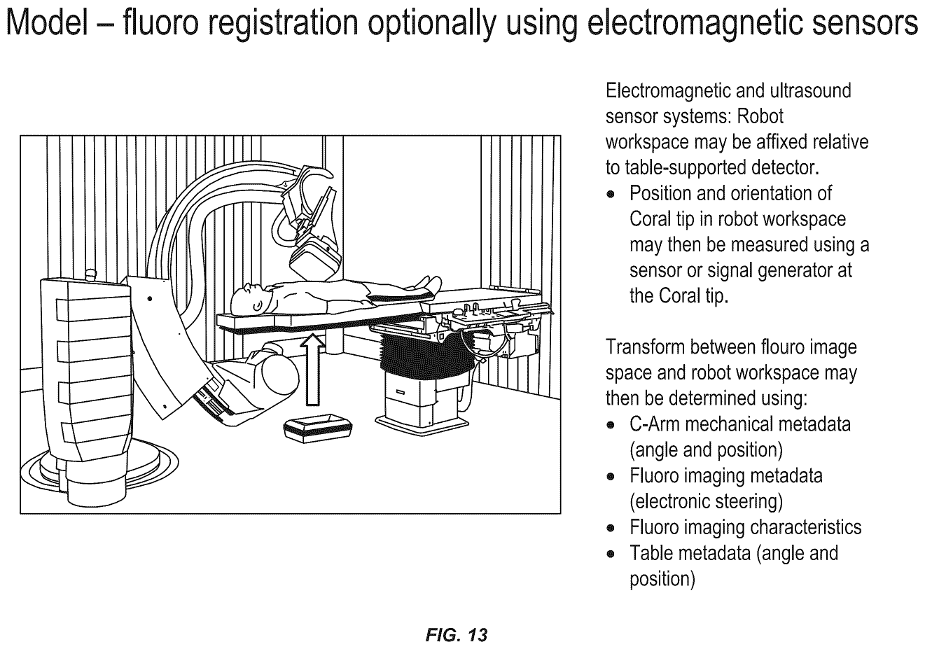

Referring now to FIGS. 12A-12F, the ultrasound-based pose module may receive a series of planar ultrasound image datasets. The ultrasound image datasets may encompass a series of cross-sections of a portion of the toolset, along with a target tissue of the patient, all within an ultrasound image field. The ultrasound pose module may determining, using the ultrasound image datasets, an ultrasound-based pose of the toolset within the ultrasound image field. This may be accomplished, for example, by identifying a centroid of the component cross-section in each scan using traditional or artificial intelligence (AI) image processing techniques. Optionally, the user can identify the cross-section at or near the end of the components. The centroids can be assembled in a 3D ultrasound image space based on the locations of the associated planes, and the pose of the component can be determined from a centerline connecting the centroids. Data on the profile and robotic-data based centerline shape may be used by the ultrasound pose module. The data process may transmit, in response to the ultrasound-based pose, a composite image including ultrasound imaging with a model of the toolset.

Referring now to FIGS. 12A and 12B tilt angle scanning from a TEE probe and associated TEE X-Plane images, respectively, can be seen. FIG. 12C is a 3D echo image space showing schematically how planar echo images from a TEE tilt angle scan can be used to determine an ultrasound-based pose of a toolset component in the 3D echo image space when the component has a lateral bend or eccentric echogenic feature. FIG. 12D schematically illustrates an alternative TEE X-Plane rotation scan which may be used in place of or in addition to the tilt angle scan of FIG. 12A to determine an ultrasound-based pose of the toolset component(s). FIGS. 12E and 12F are representative TEE X-Plane image data obtained during a tilt angle scan.

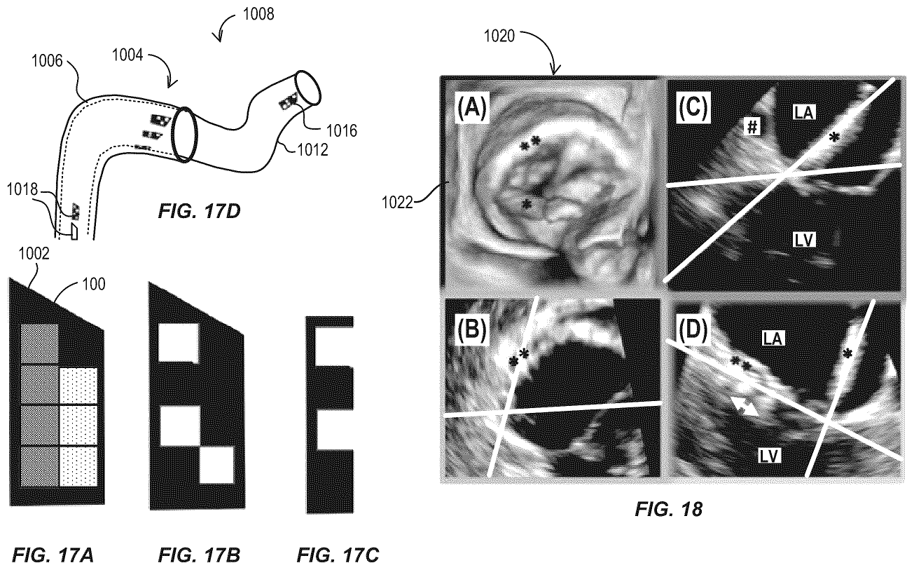

Referring now to FIGS. 17A-17D, a custom marker configuration 1002 and associated pattern 1004 may limit the arc-angle (and hence any lateral bending) of individual markers when mounted along the outer surface of a guide sheath 1006 or other cylindrical component of a multi-component interventional toolset 1008. Such markers may be formed by laser cutting tantalum sheet having a thickness of 0.001″ to 0.005″. Marker configuration 1002 has an elongate perimeter shape 1010 which can be oriented along the toolset axis to define a 1D or 2D barcode with more data squares or bits (typically 3 or more, often 4 to 10) aligned axially than circumferentially (typically 1 to 3, and ideally 1 or 2). Marker configuration 1002 may have an axial length in a range from 2.5 mm to 15 mm (typically being from 0.4 cm to 1.25 cm) and a width of from 1 mm to 4 mm. A non-symmetric (axially and laterally) perimeter shape 1010 or configuration may help identify an orientation of the marker, such as by including a boundary along only one lateral side (FIG. 17C), a boundary gap near one axial end (FIG. 17C), an angled end (FIG. 17A), a varying boundary thickness, leaving open a data bit adjacent one axial end of a continuous lateral boundary, or the like. A first plurality of data bits (such as those shown along the left side in FIGS. 17A and 17B) may define a confirmation data code and be the same for all markers of a library to avoid false maker identifications. The other data bits (along the right side) may define marker ID data and be fewer in number than those of the confirmation code. In FIGS. 17A and 17B, setting the clear bits as “1” and the blocked bits as “0” and reading from the angled end the confirmation code may be 1, 0, 1, 0, and the marker ID would be 0, 0, 1. Each data bit will preferably have a width and length greater than 2 pixels when imaged for reading, the marker preferably having a length of over 20 (and ideally over 30) pixels and a width of over 5 (ideally over 10) pixels. Libraries of less than 500 acceptable codes (often less than 200, and in many cases less than 100 or even less than 75) may be used, with any potential codes than would be read as an acceptable code if read from the back (see below) being left out of the library.

Referring to FIG. 17D, pattern 1004 of makers 1002 may include 3 or more guide sheath markers that are circumferentially offset to define a total circumferential arc angle between centers of at least 120 degrees across the pattern and no more than 45 degrees between circumferentially adjacent markers. Axially offsetting of the markers may allow reading of reversed markers on the back of a sufficiently radiolucent toolset. One or more tip marker 1016 near the distal end of a steerable sleeve 1012 extendable through a lumen 1014 of guide 1006 may facilitate feedback of robotic movement of the distal end, with the tip marker optionally comprising a split or continuous marker band. One or more proximal markers 1018 on the guide and steerable sleeve may help determine axial offset between these components, and may also allow the component pose to be determined when a radio-opaque structure of the toolset or anatomy inhibits reading of the other markers of the pattern.

Referring now to FIG. 18, an alternative echo display 1020 includes both 3D image elements 1022 and 3 planar echo images. Such multi-planar reconstruction (MPR) echo display arrangements may be registered and incorporated into a single hybrid 2D-3D display by determining the poses for each of the planar echo images and adding those planar images to the hybrid display using the multi-thread registration system described hereinbelow. The 3D image element may similarly be registered and its 2D representation (as shown in the MPR display) added in the area of the hybrid display showing the 3D model of the toolset with the 3D toolset model superimposed thereon. More details on this MPR display may be seen in an article entitled: Feasibility of a MPR-based 3DTEE guidance protocol for transcatheter direct mitral valve annuloplasty; Martin Geyer M D, et. al., 10 Aug. 2020 https://doi.org/10.1111/echo.14694

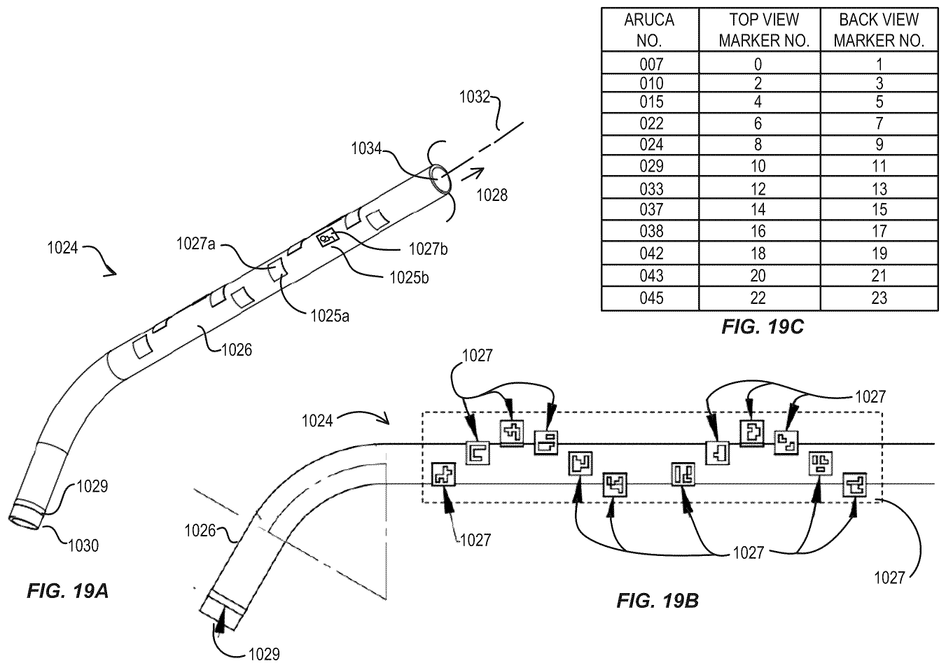

Referring to FIGS. 19A-19C, an exemplary interventional component for use with the registration techniques described herein comprises a guide sheath 1024 with an elongate flexible body 1026 having a proximal end 1028 and a distal end 1030 with an axis 1032 and lumen 1034 therebetween. The elongate body is relatively radiolucent, typically comprising a polymer laminate with braided or coiled wire reinforcement. The elongate body optionally has a bend (which may define an angle of 15-135 degrees for structural heart applications) and can be configured to be advanced distally into the patient body, for example, by recoverably receiving a straight dilator into the lumen to straighten the axis. First and second machine-readable radio-opaque markers 1025a, 1025b are disposed on the elongate flexible body, the markers each having a radially outward-facing major surface 1027a and an opposed radially-inward facing major surface 1027b. An image-based identification systema may have access to a marker library as schematically illustrated in FIG. 19C, and that library may have first and second image identification data associated with each marker. For example, a top marker ID number may be associated when an image capture device is oriented toward the outer major surface of the marker, and a back marker ID number may be associated with that same marker when the image capture device is oriented toward the inner major surfaces. Advantageously, this can allow the identification system to identify the markers regardless of which major surfaces are oriented toward the image capture device. This can facilitate the interpretation of standard (such as Aruco) or custom libraries of radio-opaque machine-readable markers regardless of the rotational orientation of the guide. As can be understood with reference to FIG. 19B, markers 1027 may be affixed to a flexible marker substrate 1027′ in the desired marker pattern and the marker substrate can then be wrapped around the guide body 1026. Note that different radio-opaque markers such as one or more continuous or split ring markers 1029 may be disposed near the distal end (optionally for image-processing-based localization of the distal tip) or may be used as a more proximal marker to identify an axial alignment of the guide. Marker patterns having some or all of these features may also be provided on a steerable sleeve passing through the lumen of the guide.

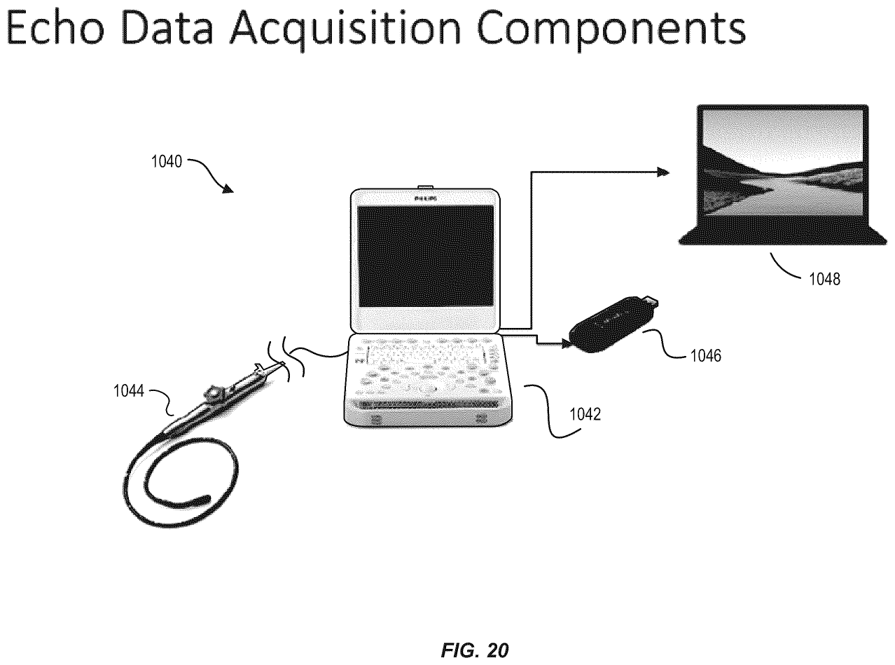

Referring to FIG. 20, an exemplary TEE system 1040 for use in the technologies described herein comprises an ultrasound machine 1042 such as a compact Philips CX50 and associated TEE probe 1044 such as an X7-2T probe. The data captured by the TEE probe can be exported in processing in at least two ways: by direct upload of DICOM data format (via USB transfer), or by live streaming of data from the ultrasound machine in MP4 format via an external capture card 1046 (screen capture), such as with an Elgato Cam Link 4K camera capture card. Alternative capture cards incorporated into a data processor of a computer 1048 running some or all of the image processing code described herein could also be used. Live streaming of the data (MP4 format via external capture card) may be preferred over standard DICOM data due to a simplified workflow (no manual upload) and greater accessibility to meta data, though in some cases additional non-standard DICOM image streaming with meta-data regarding the ultrasound acquisition settings and the like may have more advantages.

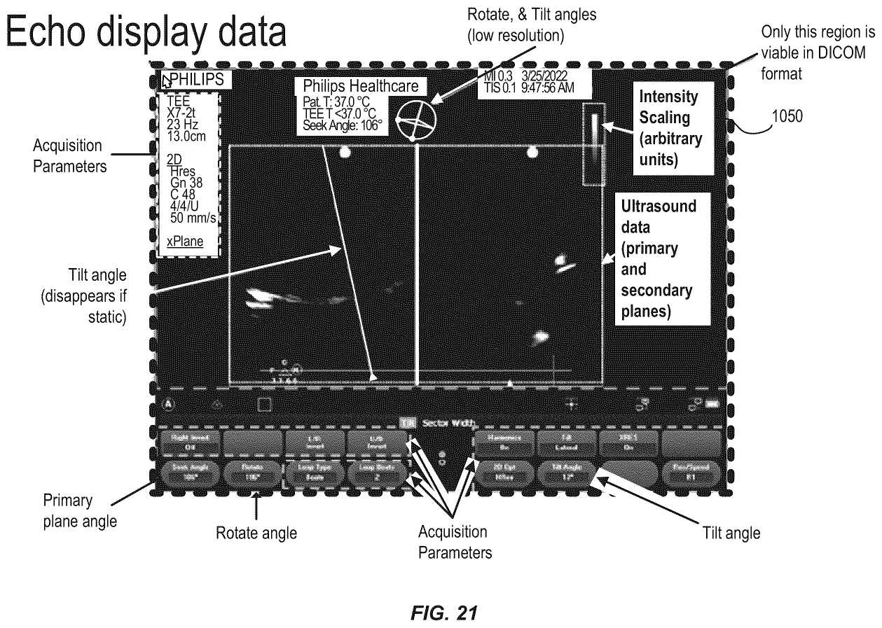

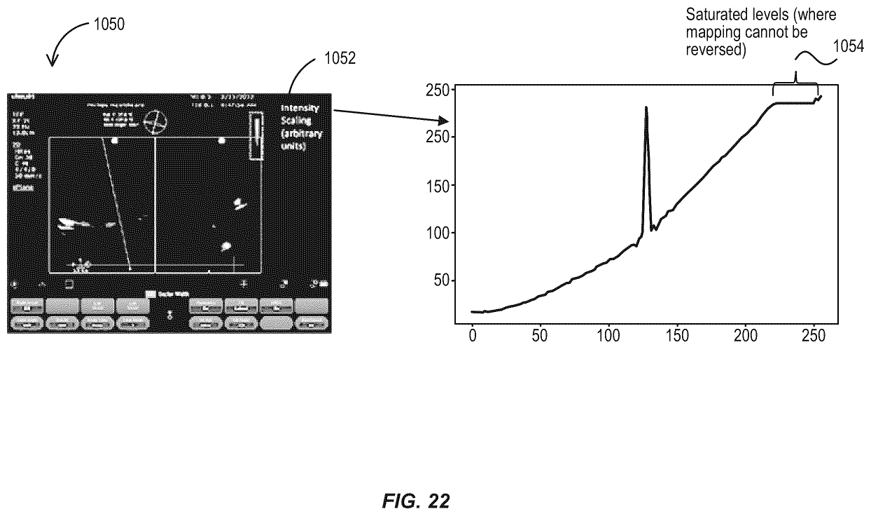

Referring to FIG. 21, information as presented to the user on a display 1050 of ultrasound system 1042 is shown in detail, with annotations. All information shown to the user is available via screen capture, including acquisition parameters that are not available in standard DICOM format. Note that rotation angle may only be available graphically in a somewhat low resolution format, and the graphical tilt angle may only be intermittently available (disappears if static for too long). As seen in FIG. 22, intensity scaling of the ultrasound pixel image data correlates well with the echo data values. A small amount of gamma correction can optionally be applied to the image greyscale pixel values. This is not severe and can be inverted (if desired) except for the saturated top end of the scale. Eliminating the saturation region 1054 would be desirable because it represents a loss of information about the echogenicity of structures with large echogenic signatures.

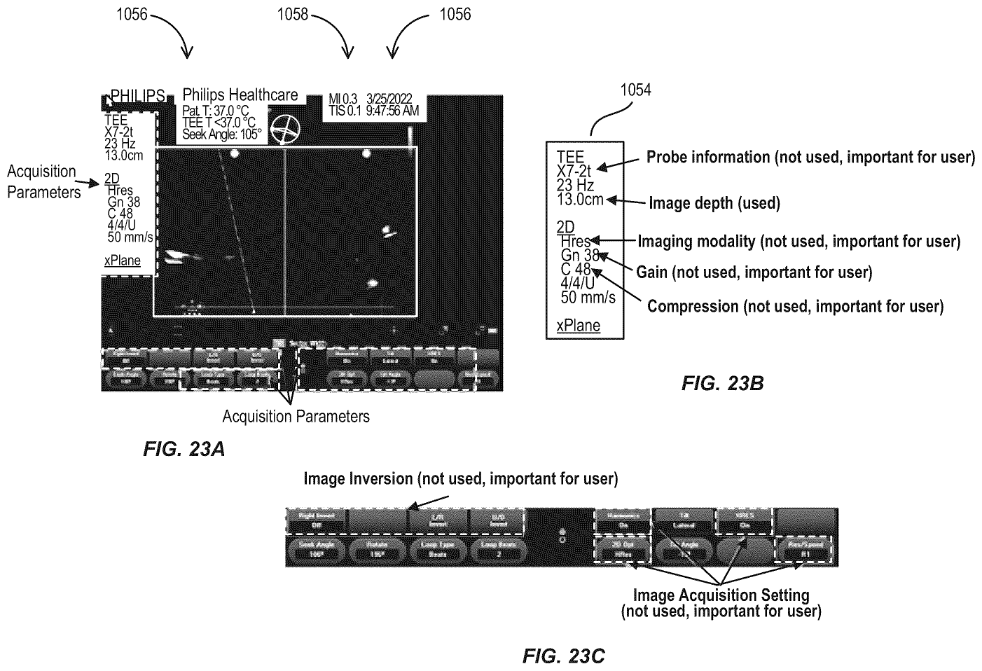

Referring to FIGS. 23A-23C, acquisition parameters 1054 identify features of the ultrasound image acquisition which are displayed and may be scanned graphically for use by the echo image processing module. Probe information, image depth, image modality, gain, and compression are examples of such acquisition parameters, and the like may be available, and the figure indicates whether such data is used or not in an exemplary embodiment of the image processing system. For example, “Res/Speed” is associated with which imaging modality is used as it affects the framerate and resolution of the scan. Image depth is used directly by the exemplary model. All other parameters shown can optionally be important for the user to know. From the overall screen capture the echo processing model crops out the regions corresponding to primary 1056 and secondary 1058 echo image planes. The echo module also extracts the intensity values for the primary and secondary planes from pixels in these regions of the screen capture. The intensity of the data reflects the relative echogenicity of the structures (optionally with additional gamma correction) and may be used by the model to reconstruct the 3D volume being imaged.

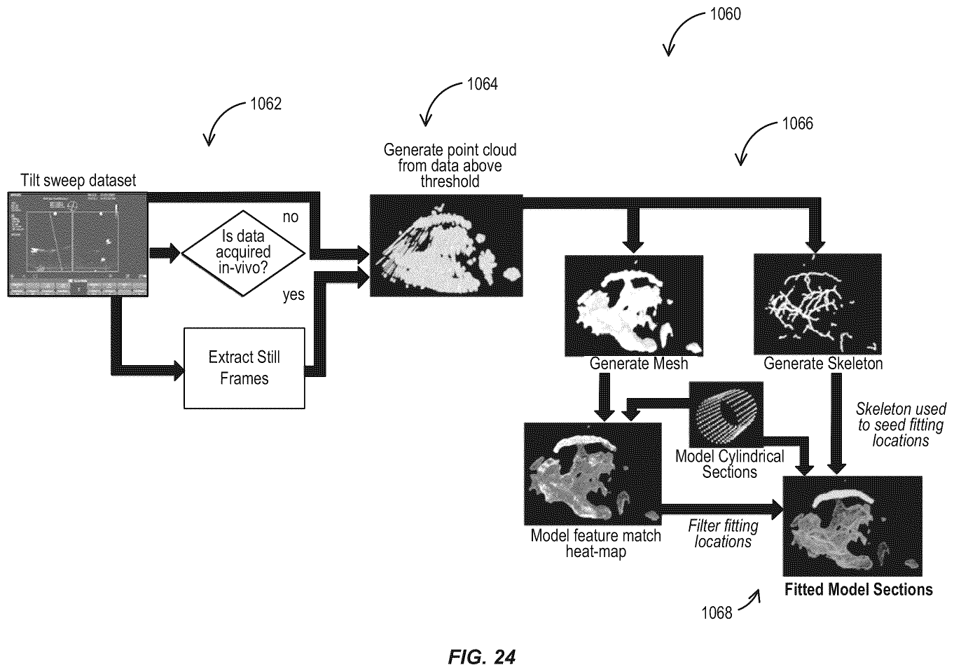

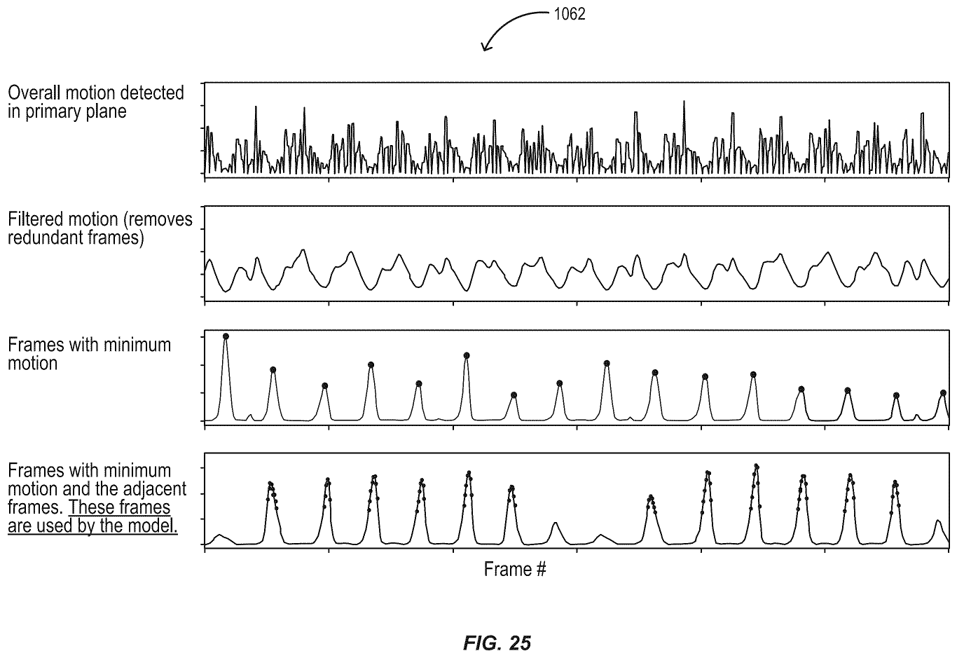

Referring to FIG. 24, a 3D echo model flow chart 1060 includes steps which can be understood with reference to the text and drawings provided herein. In general, planar echo tilt sweep data is acquired and desired quiescent planar image data extracted 1062. The data is filtered and a 3D pointcloud is generated 1064. A mesh and skeleton are generated from the point cloud 1066, and those are used to form a 3D model 1068, such as by fitting and assembling cylindrical model sections. Reviewing these steps in more detail and first addressing the extraction of quiescent image data 1062 with reference to FIG. 25, because of the motion created by a beating heart there can be a level of ambiguity in the location of the catheter. In order to overcome this ambiguity, the echo module may automatically detect relatively stationary frames, such as frames acquired during times where the aggregate motion is at/near a local minimum. Optionally, only these frames are used to generate echo-based pose data for the interventional tools. This has implications for the way in which the tilt sweep is performed, since only tilt angles that coincide with the relatively stationary frames may be used for the reconstruction.

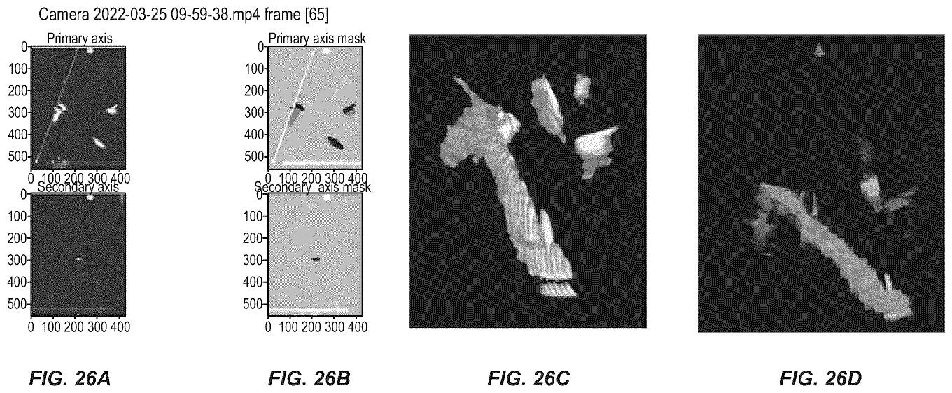

Referring to FIGS. 26A-26D, filtering of the data and generating of a point cloud 1064 can be understood. In many of the data sets significant artifacts are present in the images. It may be desirable to remove at least some of the artifacts, particularly those that are connected (or nearly connected) with the catheter (e.g., from echoes). It may be best if these artifacts are removed before generating the mesh/skeleton so that these are not affected by the artifacts. To achieve this a morphological filter (based on shape and connectivity) can be used to remove wedge-shaped features extending radially away from the probe. FIG. 26C shows an image of the 3D volume reconstruction with the raw data (white point cloud), while FIG. 26D shows coloured meshes with the artifacts removed.

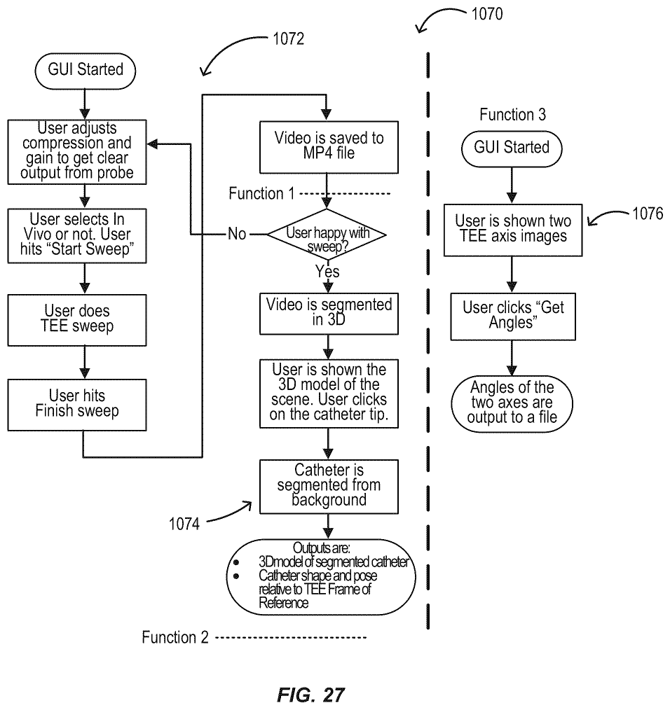

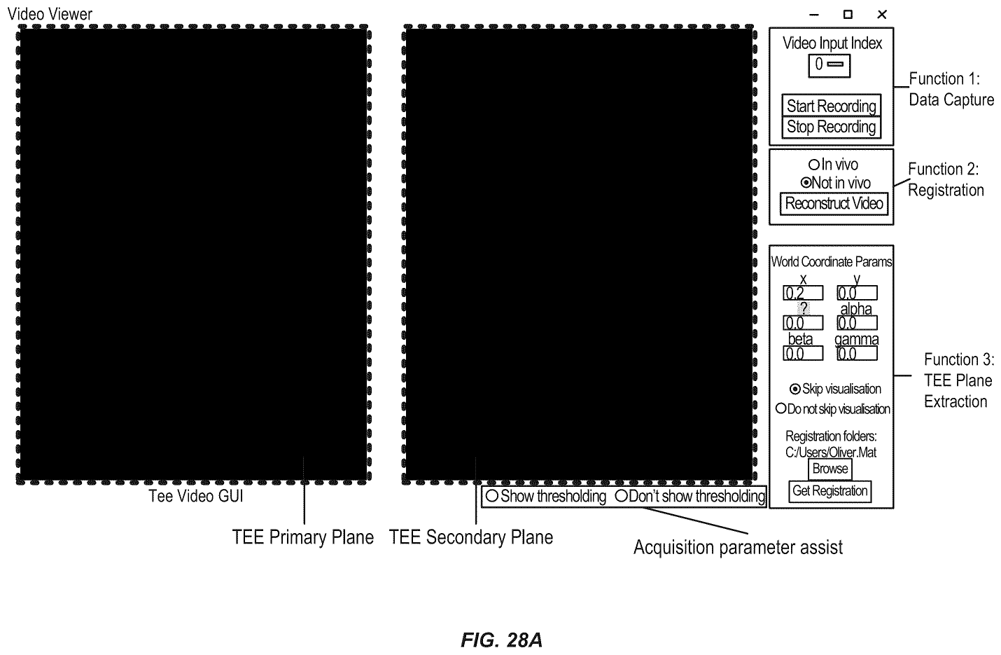

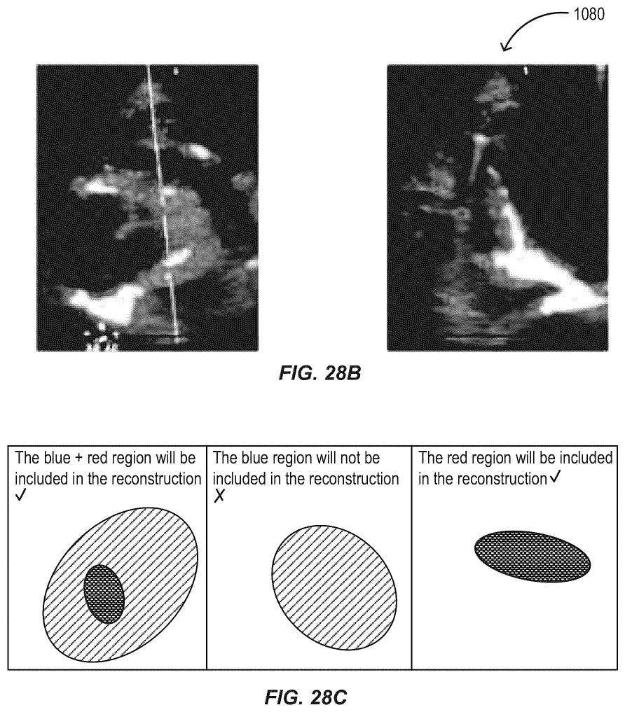

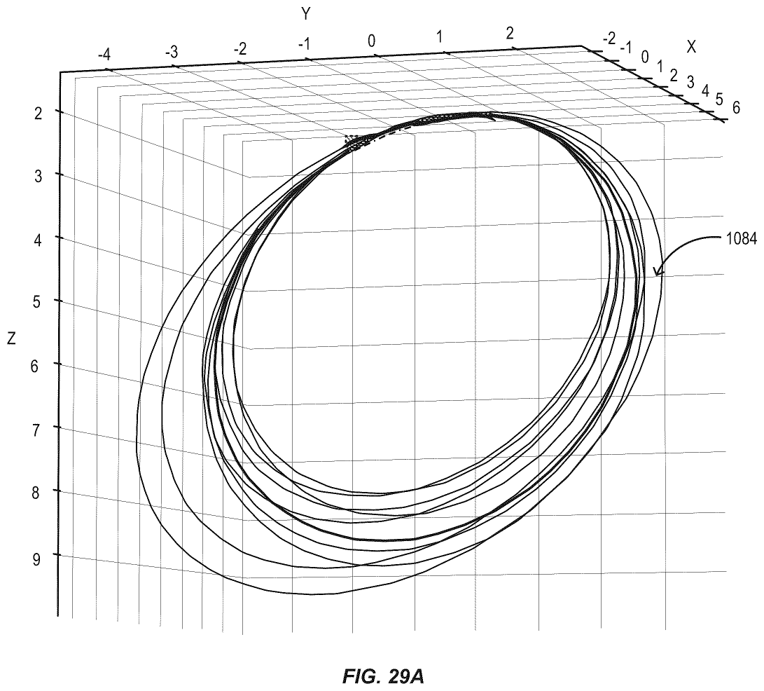

Referring to FIG. 27, a GUI flow diagram 1070 illustrates the workflow for using the echo pose generation module of the processor, as well as the main functions of the software. The first of those functions is data capture 1072, for which the GUI assists the user with setting acquisition parameters and records the data sweep. The second function is registration 1074, for which the model processes the acquired data to extract a shape & location of the catheter. The final function is plane extraction 1076, for which (after successful registration) the model will extract the two TEE planes currently displayed, and will output these planes with their location relative to the catheter. An associated GUI for this software module is shown in FIG. 28A. The assistance provided by the echo pose registration module in acquiring the echo data is illustrated in FIGS. 28B and C. Here the user is tasked with adjusting the acquisition parameters of the CX50/TEE such that the catheter shows good contrast with respect to the surrounding region. It is most beneficial that sufficient contrast 1080 is observed in the secondary plane as this is the plane that samples the cross section of the catheter along its length. Highlighting of the data in first and second colors (such as blue and red) as shown schematically in FIG. 28C indicates the extent of regions that will be included in the reconstruction (but only if the region includes some red highlighting). The aim of the user is to adjust the image acquisition so as to highlight the catheter as red & blue, and as far as practical to highlight adjacent features as black or blue with no red. Note that some connection between the catheter and the surrounding tissue is ok as long as it is isolated/localized.

Referring once again to FIGS. 27 and 28A, after a video of an echo tilt scan has been recorded a pop-up dialog box may appear asking if the user wants the model to process the video (the user can decline in cases of a poor sweep). Alternatively, the user can click “Reconstruct Video” to reconstruct a previously saved video. The user should ensure that “In vivo”/“Not in vivo” is correctly set before pressing either yes on the pop-up, or pressing “Reconstruct Video”. Progression of the model processes can be monitored on the command terminal. After the model has processes the video a screen will appear with the reconstructed 3D objects and candidate components for the catheter in red. The user can then interactively select the distal tip of the catheter from a visual examination of the 3D structure. The selected distal tip will then be highlighted, optionally in white. Controls available during tip selection may include: Select tip, Rotate view, Pan, Zoom, Reset view to primary plane, and Close window/finish.

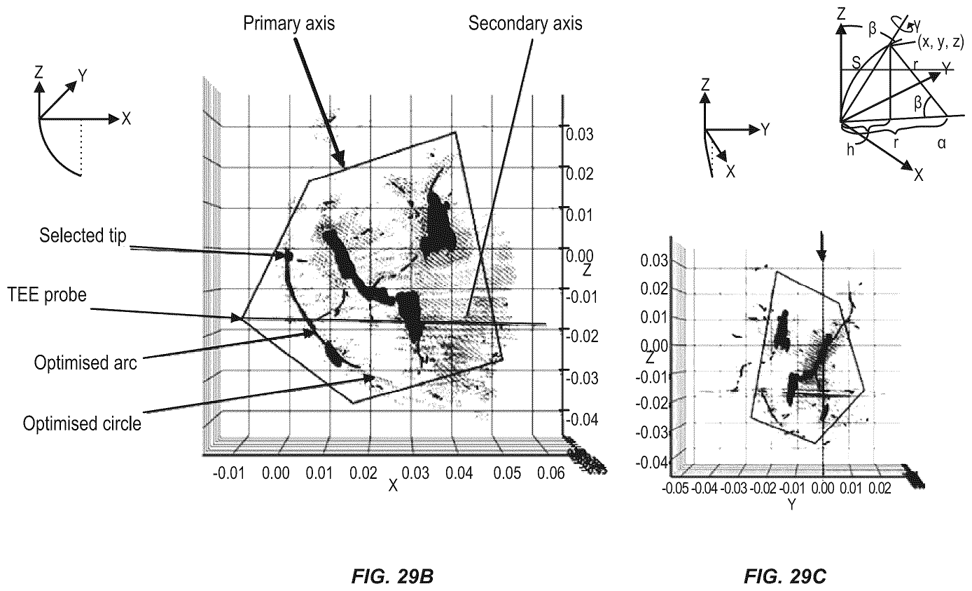

Referring now to FIGS. 29A-29C, once the user has selected the tip and exited the tip-selection window the echo pose module can then automatically begin fitting the selected object with candidate arcs 1084. The model will fit candidate arcs 1084 to the structure identified by the user to the obtained data to estimate the pose (shape and position) of the catheter. Only the section of the catheter within the TEE probe's field of view will be fitted. Different conventions may be used when fitting the catheter, and because only a limited section of the catheter is visible via the TEE probe. To do this the user may optionally input (x, y, z) (α, β, γ) pose values into the echo pose GUI. When a successful registration has been performed the user is then able to extract the current primary and secondary TEE planes and obtain their orientation relative to the catheter's position during the tilt sweep. This can be done on the most recently processed dataset or any other data set saved on the computer. World coordinate parameters may optionally be entered by the user to convert the coordinate frame from echo image space to patient or world space. The catheter pose may be estimated from the 3D reconstruction and this information can be relayed to the user for integration into other parts of the user interface. A change of reference frame can be helpful to bring the fitted catheter into the Robot Space. For example, rotation of the arc by 180° around the X-axis may be helpful. To do this the user may optionally input (x, y, z) (α, β, γ) pose values into the echo pose GUI. When a successful registration has been performed the user is then able to extract the current primary and secondary TEE planes and obtain their orientation relative to the catheter's position during the tilt sweep. This can be done on the most recently processed dataset or any other data set saved on the computer. World coordinate parameters may optionally be entered by the user to convert the coordinate frame from echo image space to patient or world space. The catheter pose may be estimated from the 3D reconstruction and this information can be relayed to the user for integration into other parts of the user interface. The arc's orientation may be aligned with the (x, y, z) (α, β, γ) robot/world pose system, and may be seated at its base position when (x, y, z) (α, β, γ)=(0, 0, 0) (0, 0, 0). FIG. 28B shows the selected tip, the position of the TEE probe, the optimized circle and associated arc of the scanned catheter, with the plot drawn in the robot/world space with the arc at its base position. In the frame extraction coordinate system, the head of the arc (nearest the selected tip) can be registered to the origin with the tangent parallel to the Z-axis. As shown in FIG. 29C, the plane in which the arc resides can be registered to Y=0.

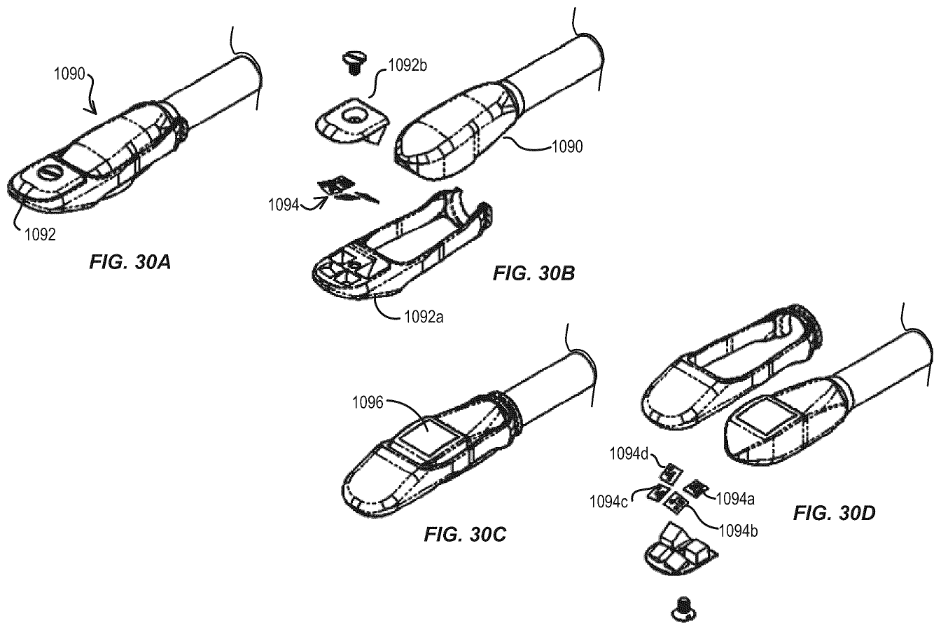

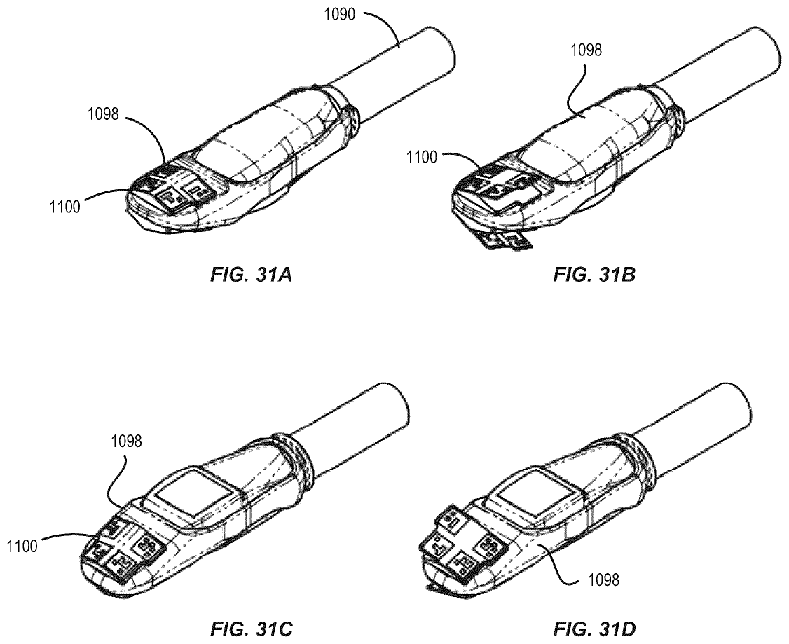

Referring now to FIGS. 30A-30D, an exemplary TEE system includes a TEE probe 1090 with a marker support 1092 affixed thereto. The marker support comprises a radiolucent material such as 3D printed polymer, extends distally of the distal end of the TEE probe, and supports a series of different Aruco markers 1094a, 1094b, 1094c, and 1094d (collectively markers 1094). The marker support is formed in two opposed portions 1092a and 1092b, and these portions constrain the markers therebetween with the markers held on planes at offset angles, which can improve pose measurements of the TEE probe using marker identification and localization. The probe support has an aperture encompassing a transducer 1096 of the probe to facilitate image acquisition. A related optical marker support structure 1098 is shown in FIGS. 31A-31D having a somewhat similar outer form factor, but may be formed in one piece. Adjacent flat surfaces of support 1098 have an angle therebetween so as to facilitate mounting a thin marker sheet 1100. Marker sheet 1100 may be formed of a pliable sheet of plastic or the like and have the markers printed thereon for camera-based pose estimation. Such flat surfaces and marker sheets may be disposed on the opposed major surfaces of the marker support 1098 so as facilitate camera image-based pose estimation from a wider range of orientations.

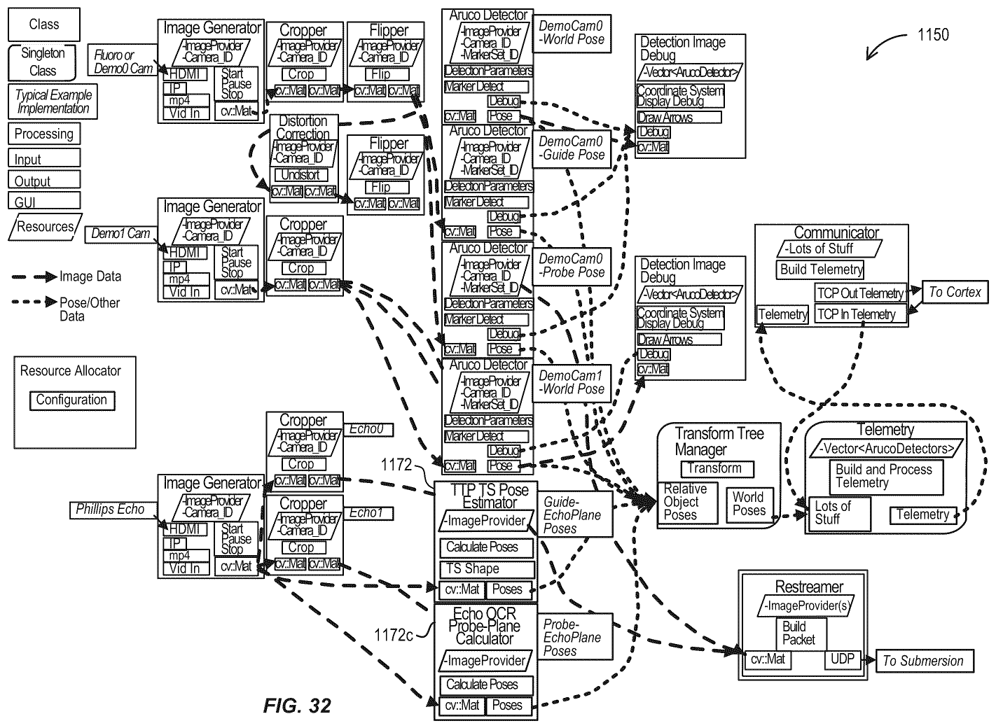

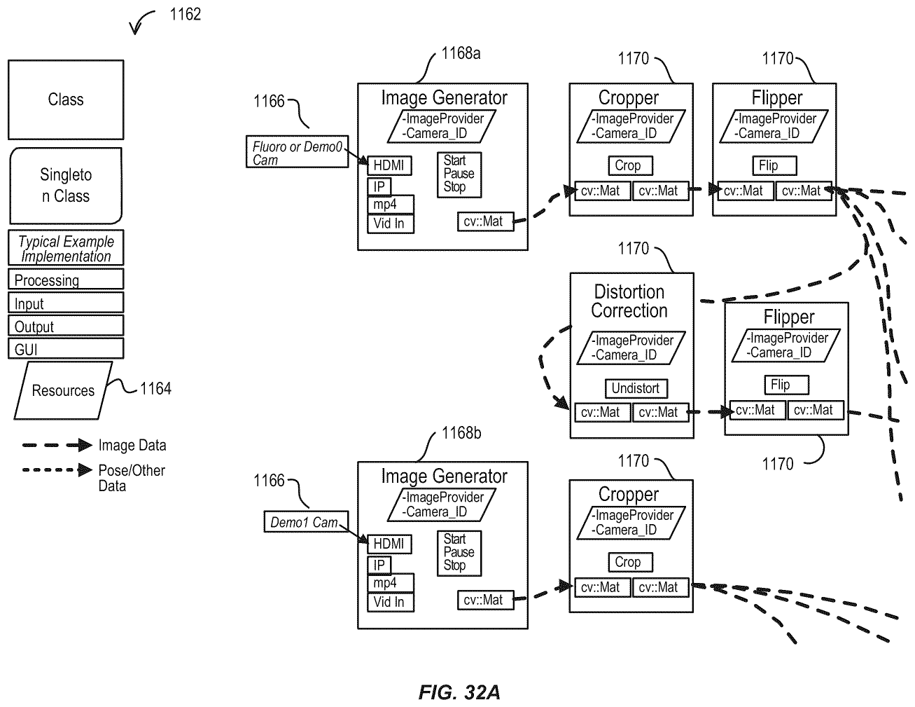

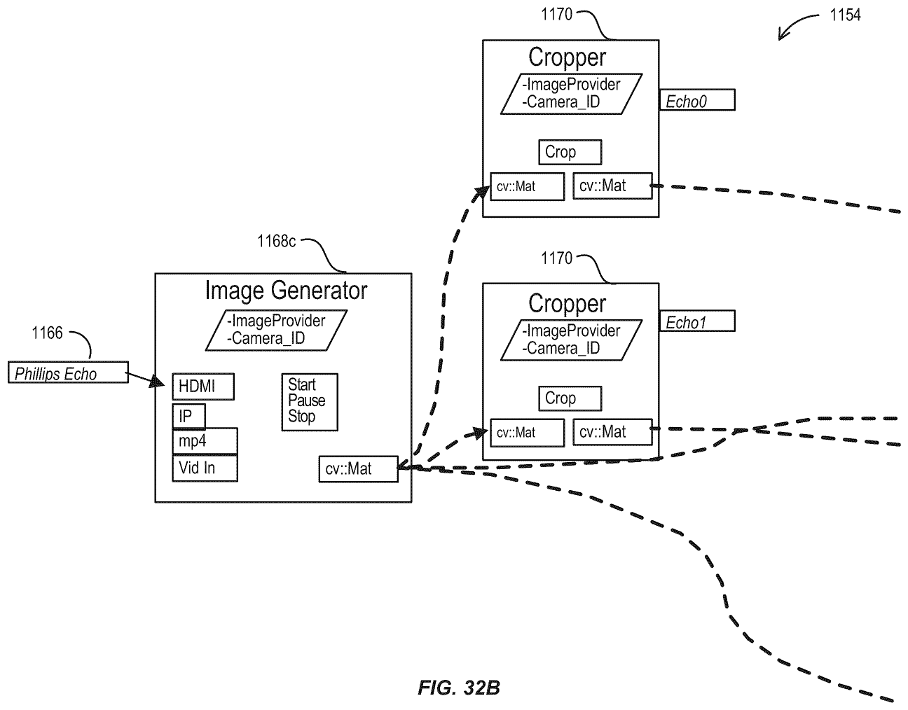

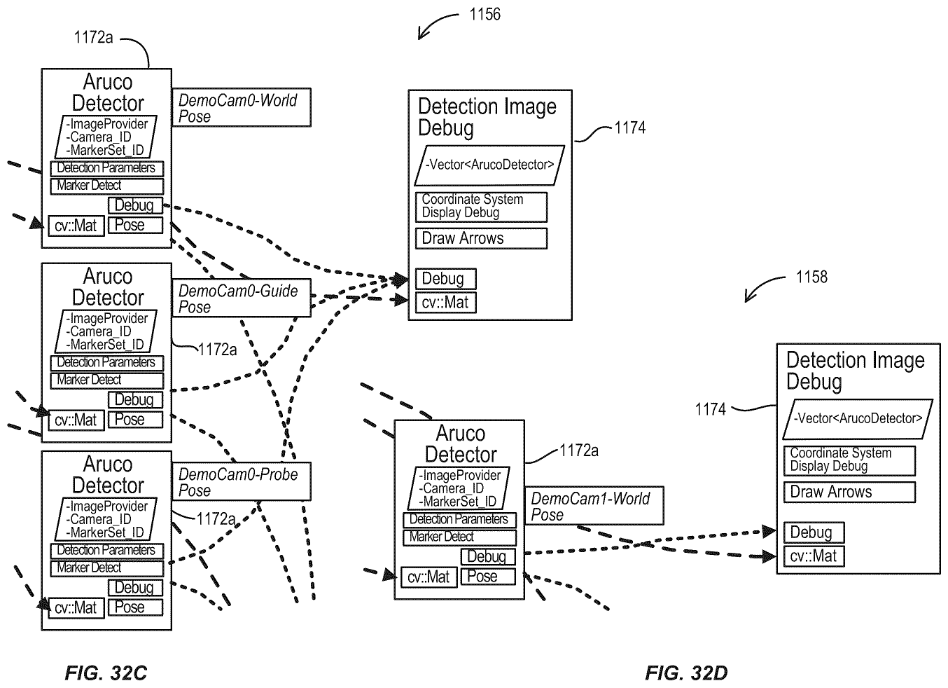

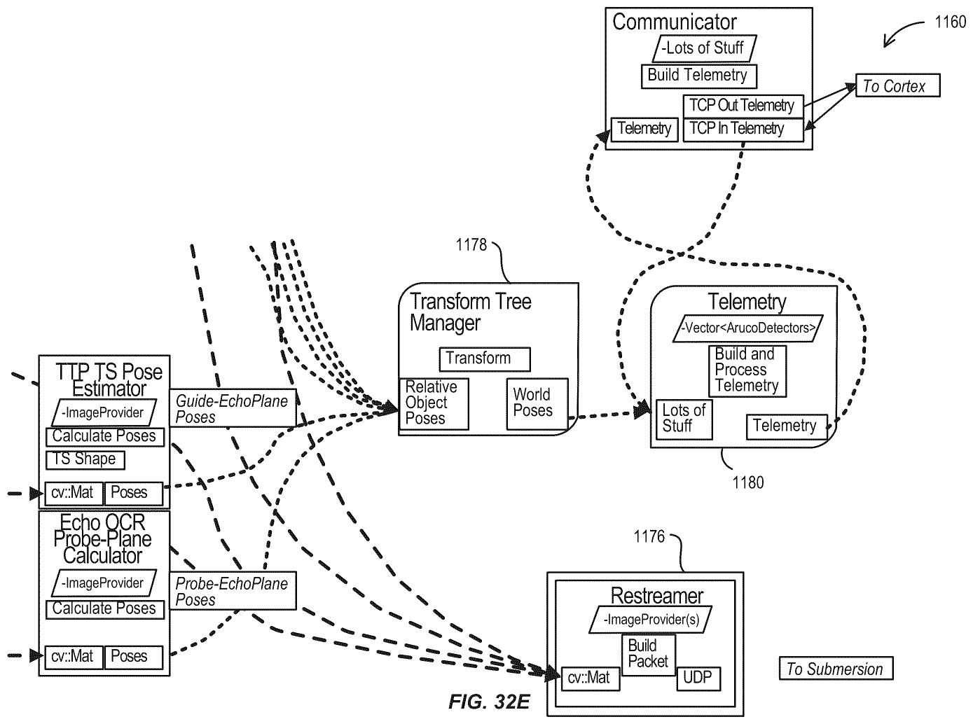

Referring now to FIGS. 32A-32E, an exemplary multi-thread registration module 1150 can accept image input from multiple image sources (including an echo image source as well as one or more additional image sources operating on different remote image modalities). In response to the image data streams, the various components of the registration module provide pose data of echo image planes acquired by the echo image source, pose data of multiple interventional components of an interventional toolset shown in those echo image planes, and modified image data streams. For convenience the components of the registration module may be referred to as sub-modules, or as modules themselves. Different portions 1152, 1154, 1158, and 1160 of registration module 1150 are shown in FIGS. 32A-32E in larger scale, and a legend 1162 is included in FIG. 32A. Note that additional resources 1164 may also be included in the overall registration module package, with two of the references of note being transformation manager data and a listing identifying the various sub-modules and data pipelines between these sub-modules of registration module 1150. The transformation manager data will include a listing of all the reference frames used throughout the overall registration module, starting with a root or patient frame (sometimes called the “world” or “robot” space). The root space is optionally defined by the pose of the fiducial marker board supported by the operating table. All other reference frames can be dependent on or “children of” (directly or indirectly) the root space so that they move when the root space moves (such as when the operating table moves). All of the parent/child relationships between the reference frames are specified in a transformation tress of the transformation manager data.

Referring now to FIGS. 32A and 32B, a series of image stream inputs 1166 receive data streams from image sources (such as an ultrasound or echo image acquisition device, a camera, a fluoroscope, or the like) for use by a series of image generators or formators 1168, including a fluoro image generator 1168a, a camera image generator 1168b, and an echo image generator 1168c. The image generators are configured to format the image stream for subsequent use, such as by converting an MP4 or other image stream format to an OpenCV Image Datastream format. Note that multiple different image generators may receive and format the same input image stream data to format or generate image data streams for different pose tasks. Regardless, the formatted image data streams can then be transmitted from the image generators 1160 to one or more image stream modifiers 1170. There are a variety of different types of image stream modifiers, including croppers (which crop or limit the image area of the input image stream to a sub-region), flippers (which flip the image data left-right, up-down, or both), distortion correctors (which apply correction functions to the pixels of the image stream to reduce distortions), and the like. A series of image stream modifiers may be used on a single image stream to, for example, crop, flip, remove distortions, and again flip the fluoro image stream data. The image modifiers may transmit modified image streams to different sub-modules, optionally applying different modifications. For example, a cropper may crop multiple regions of interest from the echo image stream, sending the primary plane to one sub-module and a region of an acquisition parameter to an optical character reader sub-module. Alternatively the cropping of separate regions of a particular input image data stream may be considered to be performed by separate croppers, as in both cases the actual code may involve running different instances of the same cropping sub-module. The output from these modification modules generally remains image data streams (albeit modified).

Referring now to FIGS. 32C, 32D, and 32E, the modified image data streams from the image modifiers are transmitted to a number of different image analyzers and augmentors 1172. These sub-modules analyze the image stream data to generate pose data and/or to augment the image streams, most often superimposing indicia of pose data on the image streams. First addressing a sub-group of the image analyzers identified as Aruco detectors 1172a, these sub-modules detect specific markers (Aruco or others) in the modified image streams they receive, and in response, transmit pose data determined from those markers. The Aruco detectors may identify only a specific set of Aruco or other markers which the registration resources list for that sub-module. Once the markers are identified, pixels at corners (or other key points) of the identified markers may determine in the 2D image plane, and the determined corners from all the identified markers may be fed into a solver having a 3D model of the interventional component. The 3D model has those corners, allowing the solver to determine a pose of the interventional component in the image stream. Aruco detectors 1172 may also transmit an image stream that has been augmented by a marker identifier/localizer, such as an outline of the marker, a small reference frame showing a center or corner of the marker and its orientation, or the like.

Referring still to FIGS. 32, another sub-group of the image analyzers comprises an echo-based toolset pose estimator (which may use echo sweeps to generate 3D point clouds and derive toolset pose data in echo space as described above). A final sub-group of the image analyzers comprises an echo probe plane pose calculator 1172c which determines a location of an echo probe (TEE or ICE), and also the poses of the image planes associated with that probe (often using OCR data). Once again, these analyzers generate both pose data and augmented image data streams with indicia of the pose data.

Referring to FIGS. 32D and 32E, the modified and/or augmented images may be transmitted to debug modules 1174 for review and development, and/or to a re-streamer 1176 that publishes the image data streams for use by the GUI module for presentation to the user (as desired), or for use by other modules of the robotic system. The pose data from all the image analyzers are transmitted to a transformation tree manager 1178 that performs transforms on the pose data per the transformation manager data (described above) so as bring some or all of the pose data into the root space (or another common frame). That pose data can then be packaged per appropriate protocols for use by other modules in a telemetry module 1180 and transmitted by a communication sub-module 1182.

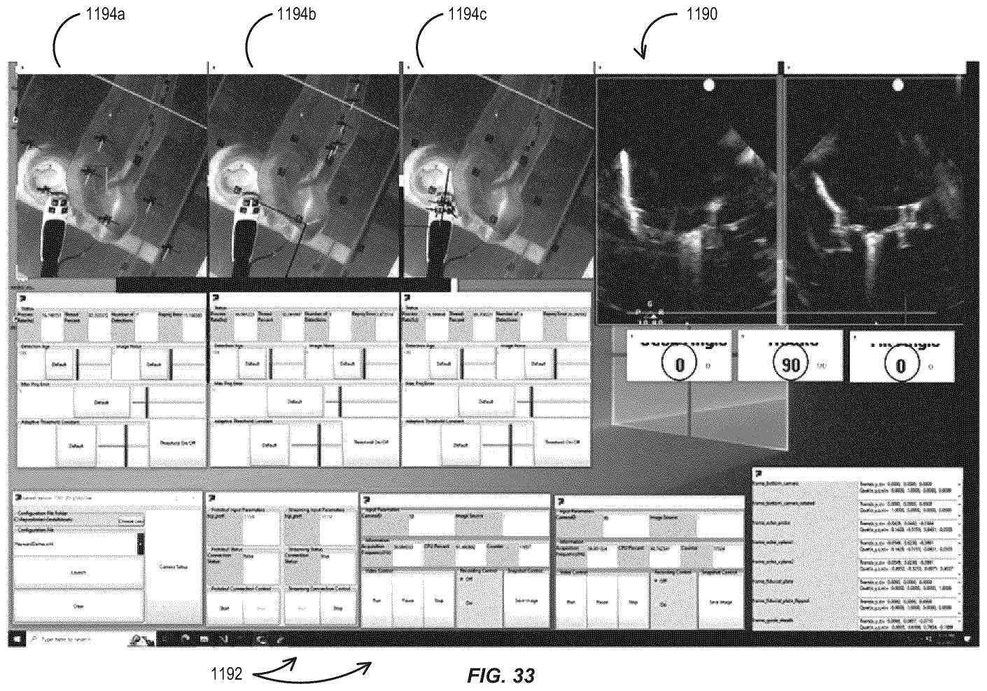

Referring now to FIG. 33, a registration module GUI 1190 includes a series of windows 1192 associated with selected sub-modules, along with a series of image displays. The sub-module windows present data on pose error projections and the like, and allow the user to vary pose generation parameters such as by introducing and varying image noise (which can be beneficial), varying pose error projection thresholds (such that low-confidence pose data may be excluded), varying thresholding parameters for image processing, or the like. The image displays may comprise augmented image displays showing, for example, a marker board image display 1194a showing identified reference frames for individual markers and the overall marker board as identified by a “world pose” Aruco detector module, a guide sheath display 1194b showing the identified frames of the individual guide sheath markers and an overall guide sheath frame, a TEE probe display 1194c showing the identified frames of the TEE probe, and so on.

While the exemplary embodiments have been described in some detail for clarity of understanding and by way of example, a variety of modifications, changes, and adaptations of the structures and methods described herein will be obvious to those of skill in the art. Hence, the scope of the present invention is limited solely by the claims attached hereto.

1. A system for a user to diagnose or treat a patient, the system for use with an ultrasound imaging probe, a fluoroscopy system, and a toolset, the probe insertable into a patient body and the toolset having a proximal end and a distal end with an axis therebetween, the distal end insertable into the patient body, the system comprising:
a data processor having:
an ultrasound input for receiving ultrasound image data generated using the probe, the ultrasound image data encompassing a distal portion of the toolset and a target tissue of the patient within an ultrasound image field;
a fluoroscopy input for receiving fluoroscopy image data generated using the fluoroscopy system, the fluoroscopy image data encompassing the distal portion of the toolset and the probe within a fluoroscopy image field, the ultrasound image data and the fluoroscopy image data comprising image data;
a pose determining module coupled with the ultrasound input and the fluoroscopy input, the pose determining module configured for determining, in response to the image data, a pose of the toolset within the patient body; and
a display coupled with the processor so as to display an image of the toolset, in response to the pose data, such that the user can guide diagnosis or treatment of the target tissue.

2. The system of claim 1, wherein the processor comprises a data processor for a multi-image-mode and/or a multi-component interventional system, the toolkit comprising a first interventional component configured for insertion into an interventional therapy site of the patient body:
the ultrasound input comprising a first input for receiving a first image data stream of the interventional therapy site, the first image data stream including image data of the first component;
the fluoroscopy input comprising a second input for receiving a second image data stream;
the processor comprising a first module, a second module, and an alignment module, the first module coupled to the first input, the first module determining a pose of the first interventional component relative to the first image data stream in response to the image data of the first component;
the second module coupled to the second input, the second module determining an alignment of the first image data stream relative to the patient body in response to the second image data stream;
the alignment module configured for determining pose data of the first component relative to the patient body from the pose of the alignment of the first image data stream and from the pose of the first interventional component relative to the first image data stream; and
the output configured for transmitting interventional guiding image data and the pose data of the first component relative to the patient data.

3. The system of claim 1, wherein the ultrasound input is configured for receiving 3D ultrasound data, the 3D ultrasound data optionally comprising a series of planar ultrasound image datasets encompassing a series of cross-sections of a portion of the toolset and the target tissue; and wherein the processor comprises an ultrasound-based pose determining module for determining, in response to the ultrasound image datasets, an ultrasound-based pose of the toolset within the ultrasound image field.

4. The system of claim 1, wherein the toolset comprises a plurality of fiducial markers, and wherein the data processor comprises a module for determining, in response to the fluoroscopic image data associated with a plurality of fiducial markers, the pose of the toolset, the fiducial markers comprising machine-readable radio-opaque markers disposed on the toolset.

5. A data processor for a multi-image-mode and/or a multi-component interventional system, the system comprising a first interventional component configured for insertion into an interventional therapy site of a patient body having a target tissue:
a first input for receiving a first image data stream of the interventional therapy site, the first image data stream including image data of the first component;
a second input for receiving a second image data stream;
a first module coupled to the first input, the first module determining a pose of the first interventional component relative to the first image data stream in response to the image data of the first component;
a second module coupled to the second input, the second module determining an alignment of the first image data stream relative to the patient body in response to the second image data stream;
an alignment module for determining pose data of the first component relative to the patient body from the pose of the alignment of the first image data stream and from the pose of the first interventional component relative to the first image data stream; and
an output for transmitting interventional guiding image data and the pose data of the first component relative to the patient data.

6. The data processor of claim 5, wherein the first image data stream comprises a planar ultrasound image data stream, and wherein the first module determines the pose data of the first interventional component relative to a 3D image data space of the ultrasound image data stream, and wherein the first image data stream comprises tilt sweep data defined by a series of image planes extending from a surface of a transesophageal echocardiography (TEE) transducer surface, tilt angles between the planes and the TEE transducer surface varying, the first module configured to extract still frames associated with the planes, assemble the still frames into a 3D point cloud of data exceeding a threshold, generate a 3D mesh and a 3D skeleton from the 3D point cloud, fit model sections based on the mesh and the skeleton, and fit a curve to the model sections to determine the pose data of the first component, the first component comprising a cylindrical catheter body having a bend.

7. The data processor of claim 5, wherein the first component has a machine-readable marker and the second image data stream comprises a fluoroscopic image data stream including image data of the marker and a pattern of machine-identifiable fiducial markers, the fiducial markers included in a marker board supported by an operating table, the second module configured to determine the pose data by determining a fluoroscope-based pose of the first component in response to the image data of the marker and the pattern of machine-identifiable fiducial makers, and wherein the pose data is indicative of a confidence of the fluoroscope-based pose.

8. The data processor of claim 5, wherein the first image data stream is generated by a first image capture device having a first imaging modality, the component being distinct in the first image data stream and the target tissue being indistinct in the first image data stream, and wherein the second image data stream is generated by a second image capture device having a second imaging modality, the component being indistinct in the second image data stream and the target tissue being distinct in the second image data stream.

9. The data processor of claim 5, wherein the first image data stream comprises a fluoroscope image data stream, the first component including a machine-readable marker, and the second image data stream comprising the fluoroscopic image data stream, the fluoroscope image data including image data of a marker included on a second component, the first and second modules comprising first and second computer vision threads identifying first and second pose data regarding the first and second components, respectively.

10. The data processor of claim 9, wherein the first interventional component comprises a robotic steerable sleeve and the second component comprises a guide sheath having a lumen, the lumen receiving the steerable sleeve axially therein.

11. The data processor of claim 9, wherein the second component comprises a TEE or ICE probe, a probe system comprising the TEE or ICE probe and an ultrasound system generating image steering data indicative of alignment of the second image data stream relative to a transducer of the TEE or ICE probe, the pose data of the first component being generated using the image steering data.

12.-13. (canceled)

14. A method for using a medical robotic system to diagnose or treat a patient, the method comprising:
calibrating fluoroscopic image data generated by a fluoroscopic image acquisition system;
determining, with a processor of the medical robotic system and in response to the calibrated fluoroscopic image data, an alignment of the fluoroscopic image data with a therapy site by imaging the therapy site and a plurality of fiducial markers using the fluoroscopic image acquisition system;
capturing toolset image data, with the fluoroscopic image acquisition system, the toolset image data encompassing a portion of a toolset of the medical robotic system in the therapy site;
calculating, with the processor of the medical robotic system and in response to the captured toolset image data and the determined alignment, a pose of the toolset in the therapy site; and
driving movement of the toolset in the therapy site using the pose.

15. The method of claim 14, wherein the captured toolset image data does not include some or all of the fiducial markers, and further comprising displacing some or all of the fiducial markers from a field of view of the fluoroscopic image acquisition system between the imaging of the therapy site and the fiducial markers and the capturing of the toolset image data.

16. The method of claim 14, wherein the portion of the toolset comprises a guide sheath having a lumen extending from a proximal end outside the patient distally to the therapy site, wherein the pose comprises a pose of the guide sheath, and wherein driving movement of the toolset comprises articulating a steerable body extending through the lumen while the guide sheath remains in the pose.

17. The method of claim 14, wherein the image capture surface of the fluoroscopic image acquisition system is disposed above the patient during use, and wherein the determining and calculating steps are performed using an optical acquisition model having a model acquisition system disposed below the patient during use.

18. The method of claim 14, further comprising, using the processor of the medical robot system:
superimposing models of the fiducial markers on the imaged therapy site based on the alignment,
comparing the superimposed models of the fiducial markers to the imaged fiducials of the fluoroscopic image data,
determining an error of the alignment, and
compensating for the error of the alignment in an image of the therapy site displayed to the user so that a model of the toolset portion superimposed on the imaged therapy site and an image of the toolset substantially correspond in the image of the therapy site.

19. The method of claim 14, wherein the fiducial markers comprise machine-readable standard or custom 2D fiducial barcode markers, and further comprising automatically identifying codes of the fiducial markers with the processor of the robotic system in response to the image data, and using the codes to determine the alignment.

20. The method of claim 14, wherein the toolset has one or more toolset fiducial markers comprising one or more machine-readable standard or custom 2D fiducial barcode marker, and further comprising automatically identifying the one or more codes of the toolset fiducial marker(s) with the processor of the robotic system in response to the image data, and using the toolset code(s) to determine the alignment.

21.-24. (canceled)
